(19) United States
(12) Patent Application Publication
(10) Pub. No.: US 2010/0168072 A1
(43) Pub. Date: Jul. 1, 2010

(54) Drug Combinations for the Treatment of Duchenne Muscular Dystrophy

(75) Inventors: Graham Michael Wynne, Abingdon (GB); Stephen Wren, Abingdon (GB); Peter Johnson, Abingdon (GB); Paul Price, Abingdon (GB); Olivier De Moor, Abingdon (GB); Gary Nugent, Abingdon (GB); Richard Storer, Abingdon (GB); Richard Joseph Pye, Abingdon (GB); Colin Richard Dorgan, Abingdon (GB)

Correspondence Address: Jones Day, 222 East 41st Street, New York, NY, 10017-6702, US

(73) Assignee: BIOMARIN IGA LIMITED

(21) Appl. No.: 12/600,240

(22) PCT Filed: Aug. 1, 2008
(86) PCT No.: PCT/GB08/50649

§ 371 Date: Mar. 22, 2010

(30) Foreign Application Priority Data
Aug. 3, 2007 (GB) 0715087.3

(51) Int. Cl. A61K 31/56; A61K 31/416; A61K 31/4192; A61K 31/454; A61K 31/5377

(52) U.S. Cl. 514/171; 514/406; 514/359; 514/322; 514/234.2

(57) ABSTRACT

Combinations comprising (or consisting essentially of) one or more compounds of the formula (I) or (II) with one or more ancillary compounds, to processes for preparing the combinations, and to various therapeutic uses of the combinations. Also provided are pharmaceutical compositions containing the combinations as well as a method of treatment of Duchenne muscular dystrophy, Becker muscular dystrophy or cachexia using the combinations.

### TECHNICAL FIELD

This invention relates to combinations comprising (or consisting essentially of) one or more compounds of the formula (I) or (II) as defined herein with one or more ancillary compounds, to processes for preparing the combinations, and to various therapeutic uses of the combinations. Also provided are pharmaceutical compositions containing the combinations as well as a method of treatment of Duchenne muscular dystrophy, Becker muscular dystrophy or cachexia using the combinations.

### BACKGROUND OF THE INVENTION

Duchenne muscular dystrophy (DMD) is a common, genetic neuromuscular disease associated with the progressive deterioration of muscle function, first described over 150 years ago by the French neurologist, Duchenne de Boulogne, after whom the disease is named. DMD has been characterized as an X-linked recessive disorder that affects 1 in 3,500 males caused by mutations in the dystrophin gene. The gene is the largest in the human genome, encompassing 2.6 million base pairs of DNA and containing 79 exons. Approximately 60% of dystrophin mutations are large insertion or deletions that lead to frameshift errors downstream, whereas approximately 40% are point mutations or small frameshift rearrangements. The vast majority of DMD patients lack the dystrophin protein. Becker muscular dystrophy is a much milder form of DMD caused by reduction in the amount, or alteration in the size, of the dystrophin protein. The high incidence of DMD (1 in 10,000 sperm or eggs) means that genetic screening will never eliminate the disease, so an effective therapy is highly desirable.

A number of natural and engineered animal models of DMD exist, and provide a mainstay for preclinical studies (Allamand, V. & Campbell, K. P. Animal models for muscular dystrophy: valuable tools for the development of therapies. Hum. Mol. Genet. 9, 2459-2467 (2000).) Although the mouse, cat and dog models all have mutations in the DMD gene and exhibit a biochemical dystrophinopathy similar to that seen in humans, they show surprising and considerable variation in terms of their phenotype. Like humans, the canine (Golden retriever muscular dystrophy and German short-haired pointer) models have a severe phenotype; these dogs typically die of cardiac failure. Dogs offer the best phenocopy for human disease, and are considered a high benchmark for preclinical studies. Unfortunately, breeding these animals is expensive and difficult, and the clinical time course can be variable among litters.

The mdx mouse is the most widely used model due to availability, short gestation time, time to mature and relatively low cost (Bulfield, G., Siller, W. G., Wight, P. A. & Moore, K. J. X chromosome-linked muscular dystrophy (mdx) in the mouse. Proc. Natl Acad. Sci. USA 81, 1189-1192 (1984)).

Since the discovery of the DMD gene about 20 years ago, varying degrees of success in the treatment of DMD have been achieved in preclinical animal studies, some of which are being followed up in humans. Present therapeutic strategies can be broadly divided into three groups: first, gene therapy approaches; second, cell therapy; and last, pharmacological therapy. Gene- and cell-based therapies offer the fundamental advantage of obviating the need to separately correct secondary defects/pathology (for example, contractures), especially if initiated early in the course of the disease. Unfortunately, these approaches face a number of technical hurdles. Immunological responses against viral vectors, myoblasts and newly synthesized dystrophin have been reported, in addition to toxicity, lack of stable expression and difficulty in delivery.

Pharmacological approaches for the treatment of muscular dystrophy differ from gene- and cell-based approaches in not being designed to deliver either the missing gene and/or protein. In general, the pharmacological strategies use drugs/molecules in an attempt to improve the phenotype by means such as decreasing inflammation, improving calcium homeostasis and increasing muscle progenitor proliferation or commitment. These strategies offer the advantage that they are easy to deliver systemically and can circumvent many of the immunological and/or toxicity issues that are related to vectors and cell-based therapies. Although investigations with corticosteroids and sodium cromoglycate, to reduce inflammation, dantrolene to maintain calcium homeostasis and clenbuterol to increase muscle strength, have produced promising results none of these potential therapies alone has yet been shown to be effective in treating DMD.

An alternative pharmacological approach is upregulation therapy. Upregulation therapy is based on increasing the expression of alternative genes to replace a defective gene and is particularly beneficial when an immune response is mounted against a previously absent protein. Upregulation of utrophin, an autosomal paralogue of dystrophin has been proposed as a potential therapy for DMD (Perkins & Davies, Neuromuscul Disord, S1: S78-S89 (2002), Khurana & Davies, Nat Rev Drug Discov 2:379-390 (2003)). When utrophin is overexpressed in transgenic mdx mice it localizes to the sarcolemma of muscle cells and restores the components of the dystrophin-associated protein complex (DAPC), which prevents the dystrophic development and in turn leads to functional improvement of skeletal muscle. Adenoviral delivery of utrophin in the dog has been shown to prevent pathology. Commencement of increased utrophin expression shortly after birth in the mouse model can be effective and no toxicity is observed when utrophin is ubiquitously expressed, which is promising for the translation of this therapy to humans. Upregulation of endogenous utrophin to sufficient levels to decrease pathology might be achieved by the delivery of small diffusible compounds.

### Ancillary Agents

A wide variety of ancillary agents find application in the combinations of the invention, as described in detail below.

### SUMMARY OF THE INVENTION

We have now found a group of compounds which upregulate endogenous utrophin in predictive screens and, thus, may be useful in the treatment of DMD.

According to the invention, we provide a combination comprising (or consisting essentially of) an ancillary agent and a compound of Formula (I) or (II)

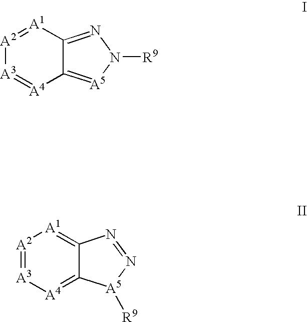

wherein
A¹, A², A³, A⁴ and A⁵, which may be the same or different, represent N or CR¹,
R₉ represents -L-R³, in which L is a single bond or a linker group and R³ represents hydrogen or a substituent and
in addition,
when an adjacent pair of A¹-A⁴ each represent CR¹, then the adjacent carbon atoms, together with their substituents may form a ring B,
when A⁵ represents CR¹, then A⁵ and N—R⁹, together with their substituents may form a ring C,
or a pharmaceutically acceptable salt thereof, optionally for the therapeutic and/or prophylactic treatment of Duchenne muscular dystrophy, Becker muscular dystrophy or cachexia.

When R⁹ represents H, compounds of formula I are tautomers of compounds of formula II.

Compounds of formula I may exist in tautomeric, enantiomeric and diastereomeric forms, all of which are included within the scope of the invention.

All of the compounds of formula I may be made by conventional methods. Methods of making heteroaromatic ring systems are well known in the art. In particular, methods of synthesis are discussed in Comprehensive Heterocyclic Chemistry, Vol. 1 (Eds.: AR Katritzky, C W Rees), Pergamon Press, Oxford, 1984 and Comprehensive Heterocyclic Chemistry 11: A Review of the Literature 1982-1995 The Structure, Reactions, Synthesis, and Uses of Heterocyclic Compounds, Alan R. Katritzky (Editor), Charles W. Rees (Editor), E. F. V. Scriven (Editor), Pergamon Pr, June 1996. Other general resources which would aid synthesis of the compounds of interest include March's Advanced Organic Chemistry: Reactions, Mechanisms, and Structure, Wiley-Interscience; 5th edition (Jan. 15, 2001).

Compounds of formula I or pharmaceutically acceptable salts thereof may be prepared from a compound of formula II

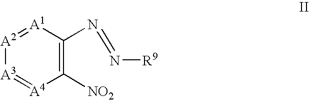

in which A¹, A², A³, and A⁴ are defined as above, in a reductive ring closure effected by reaction with thiourea-S,S-dioxide or a dithionite salt, for example an alkali metal salt, as described, for example, in EP 0 751 134. The reaction may be carried out in an aqueous solution, preferably an alcoholic aqueous solution, at a temperature of 60 to 80° C. Cyclisation will not occur in the presence of certain functionality, for example in the presence of —NH₂ or —OH functionality. These groups will need to be protected before cyclisation. For example —NH₂ groups may be protected as amides, and OH groups may be protected as ethers. Suitable protecting strategies are disclosed, for example, in EP 0 751 134.

Compounds of formula II may be prepared by a diazonium coupling reaction of a diazonium compound of formula III,

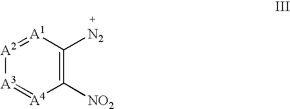

wherein A¹, A², A³, and A⁴ are defined as above, with phenyl derivatives of formula IV

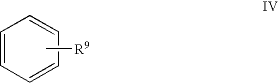

wherein R⁹ is defined as above. Conditions for the coupling are well known to the synthetic chemist. For example, reaction may take place in methanol under slightly acidic conditions, over up to 24 hours.

Compounds of formula III may be prepared by diazotisation of appropriate amines of formula V:

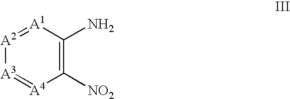

wherein A¹, A², A³, and A⁴ are defined as above. Methods of diazotisation are well known in the art, e.g. by reaction with NaNO₂/AcOH in an aqueous solution at 0 to 10° C.

Compounds of formula V may be synthesised by nitration, and subsequent deprotection, of a compound of formula VI,

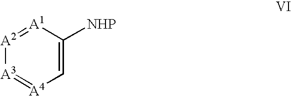

wherein A¹, A², A³, and A⁴ are as defined above and P represents a protecting group appropriate to the nitrating conditions. Nitration could be effected by, for example, cHNO₃/cH₂SO₄ in a solvent appropriate to the reaction conditions. Compounds of formulas IV and VI may be made by conventional techniques known per se. 2-Phenylindazoles of formula I can be made by a variety of processes, as outlined in the scheme below.

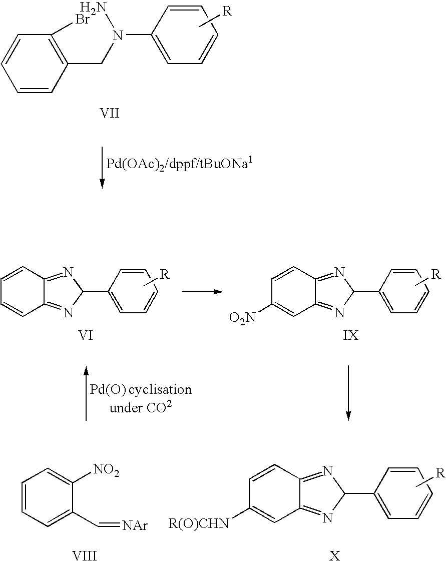

Phenyl indazoles may be made using known processes. For example hydrazines of formula VII may be cyclised using Pd (II) catalysis as described by Song, J. J. et al, Organic Letters, 2000, 2(4), 519-521.

Alternatively, phenyl indazoles of formula VII may be synthesised from an imine VIII using Pd (0) mediated cyclisation as described by Akazome, M. et al, J. Chem. Soc. Chemical Communications, 1991, 20, 1466-7.

The phenyl indazoles may then be manipulated using processes known to the skilled man. For example, nitration (as described by Elguero, J. et al, Bulletin des Societes Chimiques Beiges, 1996, 105(6), 355-358) gives nitro compound IX. The skilled man is well aware of processes by which nitro compounds may be manipulated to give a wide range of functionality. For example, reduction of the nitro compound, for example using Sn/HCl, followed by acylation, for example using an acid chloride and triethyl amine in CH₂Cl₂ gives an amide X.

In the above processes it may be necessary for any functional groups, e.g. hydroxy or amino groups, present in the starting materials to be protected, thus it may be necessary to remove one or more protective groups to generate the compound of formula I.

Suitable protecting groups and methods for their removal are, for example, those described in “Protective Groups in Organic Synthesis” by T. Greene and P. G. M. Wutts, John Wiley and Sons Inc., 1991. Hydroxy groups may, for example, be protected by arylmethyl groups such as phenylmethyl, diphenylmethyl or triphenylmethyl; acyl groups such as acetyl, trichloroacetyl or trifluoroacetyl; or as tetrahydropyranyl derivatives. Suitable amino protecting groups include arylmethyl groups such as benzyl, (R,S)-α-phenylethyl, diphenylmethyl or triphenylmethyl, and acyl groups such as acetyl, trichloroacetyl or trifluoroacetyl. Conventional methods of deprotection may be used including hydrogenolysis, acid or base hydrolysis, or photolysis. Arylmethyl groups may, for example, be removed by hydrogenolysis in the presence of a metal catalyst e.g. palladium on charcoal. Tetrahydropyranyl groups may be cleaved by hydrolysis under acidic conditions. Acyl groups may be removed by hydrolysis with a base such as sodium hydroxide or potassium carbonate, or a group such as trichloroacetyl may be removed by reduction with, for example, zinc and acetic acid.

The compounds of formula I, and salts thereof, may be isolated from their reaction mixtures using conventional techniques.

Salts of the compounds of formula I may be formed by reacting the free acid, or a salt thereof, or the free base, or a salt or derivative thereof, with one or more equivalents of the appropriate base or acid. The reaction may be carried out in a solvent or medium in which the salt is insoluble or in a solvent in which the salt is soluble, e.g. ethanol, tetrahydrofuran or diethyl ether, which may be removed in vacuo, or by freeze drying. The reaction may also be a metathetical process or it may be carried out on an ion exchange resin.

Pharmaceutically acceptable salts of the compounds of formula I include alkali metal salts, e.g. sodium and potassium salts; alkaline earth metal salts, e.g. calcium and magnesium salts; salts of the Group III elements, e.g. aluminium salts; and ammonium salts. Salts with suitable organic bases, for example, salts with hydroxylamine; lower alkylamines, e.g. methylamine or ethylamine; with substituted lower alkylamines, e.g. hydroxy substituted alkylamines; or with monocyclic nitrogen heterocyclic compounds, e.g. piperidine or morpholine; and salts with amino acids, e.g. with arginine, lysine etc, or an N-alkyl derivative thereof; or with an aminosugar, e.g. N-methyl-D-glucamine or glucosamine. The non-toxic physiologically acceptable salts are preferred, although other salts are also useful, e.g. in isolating or purifying the product.

Diastereoisomers may be separated using conventional techniques, e.g. chromatography or fractional crystallisation. The various optical isomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation.

Substituents that alkyl may represent include methyl, ethyl, butyl, eg sec butyl. Halogen may represent F, Cl, Br and I, especially Cl.

Examples of substituents that R³ in the compound of formula 1 may represent include alkyl, alkoxy or aryl, each optionally substituted by one or more, preferably one to three substituents, R₂, which may be the same or different.

In addition, compounds that may be mentioned include those of:
formula I of claim 1 or of formula II of claim 1 in which A⁵ represents N, wherein:
L is single bond and R³ represents:
thioalkyl optionally substituted by alkyl or optionally substituted aryl,
O-aryl or thioaryl, in which the aryl is optionally substituted,
optionally substituted aryl,
hydroxyl,
NR¹⁰R¹¹,
SO₂R¹²,
NR¹³SO₂R¹⁴,
C(═W)R¹⁶,
NR¹⁵C(═W)R¹⁷,
R¹⁰, R¹¹, R¹², R¹³, R¹⁴, R¹⁶ and R¹⁷, which may be the same or different, represent hydrogen, alkyl optionally substituted by optionally substituted aryl, optionally substituted aryl,
in addition,
R¹⁰ and R¹¹ together with the nitrogen to which they are attached may form a ring,
R¹² may have the same meaning as NR¹⁰R¹¹,
R¹⁶ and R¹⁷, which may be the same or different, may each represent
alkyl substituted by one or more of halogen, alkoxy optionally substituted aryl or optionally substituted aryl,
optionally substituted aryloxy,
aryl or NR¹⁰R¹¹,
and when R¹⁶ or R¹⁷ represents NR¹⁰R¹¹, one of R¹⁰ and R¹¹ may additionally represent CO alkyl optionally substituted or COaryl optionally substituted, and
in addition to the definitions shared with R¹⁷, R¹⁶ may represent hydroxyl;
or compounds of formula II of claim 1 in which A⁵ represents CH, and wherein L is single bond and R³ represents:
thioalkyl optionally substituted by alkyl or optionally substituted aryl,
thioaryl, in which the aryl is optionally substituted,
optionally substituted aryl,
hydroxyl,
NO₂,
CN,
NR¹⁰R¹¹,
halogen,
SO₂R¹²,
NR¹³SO₂R¹⁴,
C(═W)R¹⁶,
OC(═W)NR¹⁰R¹¹
NR¹⁵C(═W)R¹⁷,
R¹⁰, R¹¹, R¹², R¹³, R¹⁴, R¹⁵, R¹⁶ and R¹⁷, which may be the same or different, represent hydrogen, alkyl optionally substituted by optionally substituted aryl, optionally substituted aryl,
in addition,
R¹⁰ and R¹¹ together with the nitrogen to which they are attached may form a ring,
R¹² may have the same meaning as NR¹⁰R¹¹,
R¹⁶ and R¹⁷, which may be the same or different, may each represent
alkyl substituted by one or more of halogen, alkoxy optionally substituted aryl or optionally substituted aryl,
optionally substituted aryloxy,
aryl or NR¹⁰R¹¹,
and when R¹⁶ or R¹⁷ represents NR¹⁰R¹¹, one of R¹⁰ and R¹¹ may additionally represent CO alkyl optionally substituted or COaryl optionally substituted, and
in addition to the definitions shared with R¹⁷, R¹⁶ may represent hydroxyl.

Compounds that may be mentioned include those wherein R¹ and R², which may be the same or different, may represent:
alkyl optionally substituted by one or more halogen, alkoxy or optionally substituted aryl, thioaryl or aryloxy,
alkoxy optionally substituted by optionally by alkyl or optionally substituted aryl,
hydroxyl,
OC(═W)NR¹⁰R¹¹
aryl,
thioalkyl optionally substituted by alkyl or optionally substituted aryl,
thioaryl, in which the aryl is optionally substituted,
NO₂,
CN,
NR¹⁰R¹¹,
halogen,
SO₂R¹²,
NR¹³SO₂R¹⁴,
C(═W)R¹⁶,
NR¹⁵C(═W)R¹⁷,
P(═O)OR₄₀R₄₁,
R₁₀, R₁₁, R₁₂, R₁₃, R₁₄, R₁₅, R₁₆, R₁₇, R₄₀ and R₄₁, which may be the same or different, represent hydrogen, alkyl optionally substituted by optionally substituted aryl, optionally substituted aryl,
in addition,
NR¹⁰R¹¹ together with the nitrogen to which they are attached may form a ring,
R¹² may have the same meaning as NR¹⁰R¹¹,
when R₁₇ represents NR¹⁰R¹¹, that NR¹⁰R¹¹ may represent hydrogen, COalkyl and CO optionally substituted aryl,
R¹⁶ may represent hydroxy, alkoxy, or NR¹⁰R¹¹,
and R¹⁷ may represent alkyl substituted by one or more of halogen, alkoxy, optionally substituted aryl or NR¹⁰R¹¹.
Other compounds that may be mentioned include those of either:
formula I of claim 1 or of formula II of claim 1 in which A⁵ represents N, wherein:
L represents a linker group which is:
O, S or NR¹⁸,
alkylene, alkenylene, alkynylene, each of which may be optionally interrupted by one or more of O, S, NR¹⁸, or one or more C—C single, double or triple bonds,
and R¹⁸ represents hydrogen, alkyl, COR¹⁸.
or a compound of formula II of claim 1 in which A⁵ represents CH, wherein:
L represents a linker group which is:
O, S, NR¹⁸,
alkylene, alkenylene, alkynylene, each of which may be optionally interrupted by one or more of O, S, NR¹⁸, or one or more C—C single, double or triple bonds,
a —N—N— single or double bond,
and R¹⁸ represents hydrogen, alkyl, COR¹⁶.

Alkyl may represent any alkyl chain. Alkyl includes straight and branched, saturated and unsaturated alkyl, as well as cyclic alkyl, such as cyclopropyl, cyclobutyl, cyclopentyl, cyclohexyl and cycloheptyl. However, preferably, when any of the substituents represents alkyl, alkyl is saturated, linear or branched and has from 1 to 10 carbon atoms, preferably from 1 to 8 carbon atoms and more preferably from 1 to 6 carbon atoms. When any of the substituents represents alkyl, a particularly preferred group is cycloalkyl, for example cyclopropyl, cyclobutyl, cyclopentyl, cyclohexyl and cycloheptyl.

Aryl may represent any aromatic system. Preferably, in the compounds of formula I, aryl is an aromatic hydrocarbon or a 5 to 10 membered aromatic heterocycle containing 1 to 4 hetero atoms selected from an oxygen atom, a sulphur atom and a nitrogen atom as a ring constituent besides carbon. We prefer heterocycles which contain one or two heteroatoms. Aromatic heterocycles that may be mentioned include furan, thiophene, pyrrole, and pyridine. Particularly preferably, when aryl is an aromatic hydrocarbon, aryl represents a 6 to 10 membered monocyclic or bicyclic system, for example phenyl or naphthalene.

Saturated and unsaturated heterocycles that may be mentioned include those containing 4 to 7 ring atoms, preferably 5 or 6 ring atoms, preferably containing one to two heteroatoms selected from N, S and O. Heterocycles that may be mentioned include pyrrolidine, piperidine, tetrahydrofuran, piperazine and morpholine. N-containing heterocycles are particularly preferred, eg when NR¹⁰R¹¹ forms a heterocyclic ring.

As detailed above, when an adjacent pair of A¹-A⁴ each represent CR¹, the adjacent carbon atoms, together with their substituents may form a ring B. Also, when A⁵ represents CR¹, then A⁵ and CR¹ together with their substituents may form a ring C. Preferably ring B and/or ring C is a saturated or unsaturated 3 to 10 membered carbocylic or heterocyclic ring. Particularly preferably ring B is benzene ring. Particularly preferably ring C is a 3-10 membered saturated or unsaturated heterocyclic ring.

We particularly prefer compounds in which at least one R₁ represents NR¹⁵C(═W)R¹⁷, most particularly the group NR¹⁵COR¹⁷. We also prefer compounds in which at least one R¹ represents CONR¹⁰R¹¹.

For one group of particularly preferred compounds at least one R₁ represents an amide group NHCOR¹⁷, wherein R¹⁷ is selected from:
alkyl C₁-C₆,
alkyl C₁-C₆ substituted by phenyl
alkyl C₁-C₆ substituted by alkoxy C₁-C₆,
haloalkyl C₁-C₆,
perfluoroalkyl C₁-C₆₁
phenyl optionally substituted by one or more of halogen, alkyl C₁-C₆, alkoxy C₁-C₆, amino, (alkyl C₁-C₆)amino, di(alkyl C₁-C₆) amino or phenyl,
CH:CH phenyl,
naphthyl, pyridinyl, thiophenyl and furanyl.

We prefer compounds in which one or both of R¹ and R² are other than —COOH.

For another group of particularly preferred compounds at least one R¹ represents a group NR¹⁵CONR¹⁰R¹¹, then in which R¹⁰ and R¹¹, which may be the same or different, are selected from optionally substituted aryl, alkyl and COaryl optionally substituted. A particularly preferred group which at least one of R¹ may represent is NHCONHR¹⁵ and R¹⁵ is selected from phenyl, alkyl C₁ to C₆ and COphenyl optionally substituted by one or more halogen.

For another group of particularly preferred compounds at least one R¹ represents alkyl C₁ to C₆, optionally substituted by phenyl or a 5 or 6-membered saturated or unsaturated heterocycle containing one to two heteroatoms selected from N, S and O. Preferred heterocycles include thiophene, furan, pyridine and pyrrole.

For another group of particularly preferred compounds at least one R¹ represents COR¹⁶ and R¹⁶ is alkoxy C₁-C₆, amino, (alkyl C₁-C₆)amino or di(alkyl C₁-C₆) amino.

For another group of particularly preferred compounds at least one R₁ represents:
NO₂,
halogen,
amino or (alkyl C₁-C₆)amino or di(alkyl C₁-C₆) amino in which the alkyl C₁ to C₆ is optionally substituted by phenyl or a 5 or 6 membered saturated or unsaturated heterocycle,
NHSO₂alkyl C₁-C₆, NHSO₂-phenyl,
SO₂alkyl C₁-C₆,
phenyl optionally substituted by C₁ to C₆ alkoxy C₁-C₆,
a 5-10 membered, saturated or unsaturated, mono- or bi-cyclic heterocycle containing from 1-3 heteroatoms selected from N, S and O.

There is also wide scope for variation of the group R³. Preferably R³ represents aryl and is optionally substituted by one to three substituents, R², which may be the same or different. Particularly preferably, R³ is a 5-10 membered aromatic mono- or bi-cyclic system, especially a hydrocarbon 5-10 membered aromatic mono- or bi-cyclic system, for example benzene or naphthalene.

Alternatively, the 5-10 membered aromatic mono- or bi-cyclic system, may be a heterocyclic system containing up to three heteroatoms selected from N, O and S, for example a thiophene, furan, pyridine or pyrrole.

Preferably the substituent(s) R² is/are selected from:
alkyl C₁-C₆, optionally substituted by thiophenyl or phenoxy, each optionally substituted by halogen,
alkoxy C₁-C₆
phenyl,
thioalkyl C₁-C₆
thiophenyl, optionally substituted by halogen,
NO₂,
CN
NR¹⁰R¹¹, in which R¹⁰ and R¹¹, which may be the same or different represent hydrogen, alkyl C₁-C₆, or together with the nitrogen to which they are attached form a 5 to 7 membered ring which may contain one or more additional heteroatoms selected from N, O and S,
halogen
SO₂R¹², in which R¹² represents a 5 to 7 membered ring which may contain one or more additional heteroatoms selected from N, O and S
NHCOR¹⁷, in which R¹⁷ represents
   alkyl C₁-C₆, optionally substituted by:
      phenyl or halogen, or
      phenyl optionally substituted by alkoxy C₁-C₆, carboxy or halogen, or
      a 5 or 6 membered saturated or unsaturated heterocycle,
   phenyl or a 5 or 6 membered saturated or unsaturated heterocycle optionally substituted by halogen, alkoxy C₁ to C₆, carboxy or a group SO₂NR¹⁰R¹¹,

Particularly preferably when R² represents NR¹⁰R¹¹, NR¹⁰R¹¹ represents N-pyrrole, N-piperidine, N′(C₁-C₆ alkyl N piperazine or N-morpholine.

Preferably the linker group L represents:
—NH.NH—
—CH═CH—,
—C≡C—, or
—NCOR¹⁶ in which R¹⁶ represents phenyl or a 5 or 6 membered saturated or unsaturated heterocycle optionally substituted by halogen, alkoxy C₁ to C₆, carboxy.

A¹-A⁴ may represent N or CR¹. Consequently, the six membered ring may contain 1, 2, 3 or 4 nitrogen atoms. Embodiments of the invention exist in which two of A¹-A⁴ represent nitrogen, one of A¹-A⁴ represents nitrogen and in which all of A¹-A⁴ represents CR¹.

In a particularly preferred group of compounds:

A¹, A², A³, A⁴ and A⁵ which may be the same or different, represent N or CR¹,
R⁹ represents -L-R³, in which L is a single bond or a linker group,
either the compound is of formula I or of formula II wherein A⁵ represents N, and
L is single bond and R³ represents:
thioalkyl optionally substituted by alkyl or optionally substituted aryl,
thioaryl, in which the aryl is optionally substituted,
optionally substituted aryl,
hydroxyl,
NR¹⁰R¹¹,
SO₂R¹²,
NR¹³SO₂R¹⁴,
C(═W)R¹⁶,
NR¹⁵C(═W)R¹⁷,
R¹⁰, R¹¹, R¹², R¹³, R¹⁴, R¹⁶ and R¹⁷, which may be the same or different, represent hydrogen, alkyl optionally substituted by optionally substituted aryl, optionally substituted aryl,
in addition,
R¹⁰ and R¹¹ together with the nitrogen to which they are attached may form a ring,
R¹² may have the same meaning as NR¹⁰R¹¹,
R¹⁶ and R¹⁷, which may be the same or different, may each represent
alkyl substituted by one or more of halogen, alkoxy optionally substituted aryl or optionally substituted aryl,
optionally substituted aryloxy,
aryl or NR¹⁰R¹¹,
and when R¹⁶ or R¹⁷ represents NR¹⁰R¹¹, one of R¹⁰ and R¹¹ may additionally represent CO alkyl optionally substituted or COaryl optionally substituted, and
in addition to the definitions shared with R¹⁷, R¹⁶ may represent hydroxyl;
or the compound is of formula II in which A⁵ represents CH, and wherein L is single bond and R³ represents:
thioalkyl optionally substituted by alkyl or optionally substituted aryl,
thioaryl, in which the aryl is optionally substituted,
optionally substituted aryl,
hydroxyl,
NO₂,
CN,
NR¹⁰R¹¹,
halogen,
SO₂R¹²,
NR¹³SO₂R¹⁴,
C(═W)R¹⁶,
OC(═W)NR¹⁰R¹¹
NR¹⁵C(═W)R¹⁷,
R¹⁰, R¹¹, R¹², R¹³, R¹⁴, R¹⁵, R¹⁶ and R¹⁷, which may be the same or different, represent hydrogen, alkyl optionally substituted by optionally substituted aryl, optionally substituted aryl,
in addition,
R¹⁰ and R¹¹ together with the nitrogen to which they are attached may form a ring,
R¹² may have the same meaning as NR¹⁰R¹¹,
R¹⁶ and R¹⁷, which may be the same or different, may each represent
alkyl substituted by one or more of halogen, alkoxy optionally substituted aryl or optionally substituted aryl,
optionally substituted aryloxy,
aryl or NR¹⁰R¹¹,
and when R¹⁶ or R¹⁷ represents NR¹⁰R¹¹, one of R¹⁰ and R¹¹ may additionally represent CO alkyl optionally substituted or COaryl optionally substituted, and in addition to the definitions shared with R¹⁷, R¹⁶ may represent hydroxyl and in addition,
R¹ and R², which may be the same or different, represent:
alkyl optionally substituted by one or more halogen, alkoxy or optionally substituted aryl, thioaryl or aryloxy,
alkoxy optionally substituted by optionally by alkyl or optionally substituted aryl,
hydroxyl,
OC(═W)NR¹⁰R¹¹
aryl,
thioalkyl optionally substituted by alkyl or optionally substituted aryl,
thioaryl, in which the aryl is optionally substituted,
NO₂,
CN,
NR¹⁰R¹¹,
halogen,
SO₂R¹²,
NR¹³SO₂R¹⁴,
C(═W)R¹⁶,
NR¹⁵C(═W)R¹⁷,
R¹⁰, R¹¹, R¹², R¹³, R¹⁴, R¹⁵, R¹⁶ and R¹⁷, which may be the same or different, represent hydrogen, alkyl optionally substituted by optionally substituted aryl, optionally substituted aryl,
in addition,
NR¹⁰R¹¹ together with the nitrogen to which they are attached may form a ring,
R¹² may have the same meaning as NR¹⁰R¹¹,
when R₁₇ represents NR¹⁰R¹¹, that NR¹⁰R¹¹ may represent hydrogen, COalkyl and CO optionally substituted aryl,
R¹⁶ may represent hydroxy, alkoxy, or NR¹⁰R¹¹,
and R¹⁷ may represent alkyl substituted by one or more of halogen, alkoxy, optionally substituted aryl or NR¹⁰R¹¹.
   when an adjacent pair of A₁-A₄ each represent CR₁, then the adjacent carbon atoms, together with their substituents may form a ring B, or a pharmaceutically acceptable salt thereof, optionally for the therapeutic and/or prophylactic treatment of Duchenne muscular dystrophy, Becker muscular dystrophy or cachexia.

We also provide a method for the treatment or prophylaxis of Duchenne muscular dystrophy, Becker muscular dystrophy or cachexia in a patient in need thereof, comprising administering to the patient an effective amount of a combination of the invention.

### GENERAL PREFERENCES AND DEFINITIONS

The combinations of the invention may produce a therapeutically efficacious effect relative to the therapeutic effect of the individual compounds when administered separately.

The term “efficacious” includes advantageous effects such as additivity, synergism, reduced side effects, reduced toxicity, increased time to disease progression, increased time of survival, sensitization or resensitization of one agent to another, or improved response rate. Advantageously, an efficacious effect may allow for lower doses of each or either component to be administered to a patient, thereby decreasing the toxicity of chemotherapy, whilst producing and/or maintaining the same therapeutic effect.

A “synergistic” effect in the present context refers to a therapeutic effect produced by the combination which is larger than the sum of the therapeutic effects of the components of the combination when presented individually.

An “additive” effect in the present context refers to a therapeutic effect produced by the combination which is larger than the therapeutic effect of any of the components of the combination when presented individually.

A “pharmaceutical composition” is a solid or liquid composition in a form, concentration and level of purity suitable for administration to a patient (e.g. a human or animal patient) upon which administration it can elicit the desired physiological changes. Pharmaceutical compositions are typically sterile and/or non-pyrogenic. The term non-pyrogenic as applied to the pharmaceutical compositions of the invention defines compositions which do not elicit undesirable inflammatory responses when administered to a patient.

As used herein, the terms mobilizing agent and mobilization are terms of art referring to agents and treatments which serve to promote the migration of CD34⁺, stem, progenitor and/or precursor cells from the marrow to the peripheral blood (for a review, see e.g. Cottler-Fox et al. (2003) Stem cell mobilization Hematology: 419-437). Current standard agents for mobilization suitable for use according to the invention include G-CSF (Filgrastim™, Amgen), GM-CSF (Sargramostim™, Berlex, Richmond, Calif.) and erythropoietin (which has some mobilizing activity w.r.t. CD34⁺ cells). Alternative agents include stem cell factor (SCF) (which is particularly effective when used in combination with G-CSF) and various derivatives of G-CSF (Pegfilgrastim™, Amgen) and erythropoietin (Darbopoietin®, Amgen). The latter agents benefit from extended half-lives and so increase the temporal window available for collection. AMD3100 (AnorMed™, Vancouver, Canada), which is a reversible inhibitor of the binding of stromal derived factor (SDF-1a) to its cognate receptor CXCR4, is currently in clinical trials as a mobilizing agent. Other agents include docetaxel (see e.g. Prince et al. (2000) Bone Marrow Transplantation 26: 483-487).

The term “upregulation of utrophin” as used herein includes elevated expression or over-expression of utrophin, including gene amplification (i.e. multiple gene copies) and increased expression by a transcriptional effect, and hyperactivity and activation of utrophin, including activation by mutations. The term “utrophin upregulating agent” is to be interpreted accordingly. Thus, upregulation of utrophin covers increasing utrophin activity at the level of the encoding DNA as well as the transcriptional, translational or post-translational level. Preferred compounds of formula (I) and (II) are utrophin upregulators (as disclosed herein).

As used herein, the term “combination”, as applied to two or more compounds and/or agents (also referred to herein as the components), is intended to define material in which the two or more compounds/agents are associated. The terms “combined” and “combining” in this context are to be interpreted accordingly.

The association of the two or more compounds/agents in a combination may be physical or non-physical. Examples of physically associated combined compounds/agents include:
compositions (e.g. unitary formulations) comprising the two or more compounds/agents in admixture (for example within the same unit dose);
compositions comprising material in which the two or more compounds/agents are chemically/physicochemically linked (for example by crosslinking, molecular agglomeration or binding to a common vehicle moiety);
compositions comprising material in which the two or more compounds/agents are chemically/physicochemically co-packaged (for example, disposed on or within lipid vesicles, particles (e.g. micro- or nanoparticles) or emulsion droplets);
pharmaceutical kits, pharmaceutical packs or patient packs in which the two or more compounds/agents are co-packaged or co-presented (e.g. as part of an array of unit doses);

Examples of non-physically associated combined compounds/agents include:
material (e.g. a non-unitary formulation) comprising at least one of the two or more compounds/agents together with instructions for the extemporaneous association of the at least one compound/agent to form a physical association of the two or more compounds/agents;
material (e.g. a non-unitary formulation) comprising at least one of the two or more compounds/agents together with instructions for combination therapy with the two or more compounds/agents;
material comprising at least one of the two or more compounds/agents together with instructions for administration to a patient population in which the other(s) of the two or more compounds/agents have been (or are being) administered;
material comprising at least one of the two or more compounds/agents in an amount or in a form which is specifically adapted for use in combination with the other(s) of the two or more compounds/agents.

As used herein, the term “combination therapy” is intended to define therapies which comprise the use of a combination of two or more compounds/agents (as defined above). Thus, references to “combination therapy”, “combinations” and the use of compounds/agents “in combination” in this application may refer to compounds/agents that are administered as part of the same overall treatment regimen. As such, the posology of each of the two or more compounds/agents may differ: each may be administered at the same time or at different times. It will therefore be appreciated that the compounds/agents of the combination may be administered sequentially (e.g. before or after) or simultaneously, either in the same pharmaceutical formulation (i.e. together), or in different pharmaceutical formulations (i.e. separately). Simultaneously in the same formulation is as a unitary formulation whereas simultaneously in different pharmaceutical formulations is non-unitary. The posologies of each of the two or more compounds/agents in a combination therapy may also differ with respect to the route of administration.

As used herein, the term “pharmaceutical kit” defines an array of one or more unit doses of a pharmaceutical composition together with dosing means (e.g. measuring device) and/or delivery means (e.g. inhaler or syringe), optionally all contained within common outer packaging. In pharmaceutical kits comprising a combination of two or more compounds/agents, the individual compounds/agents may unitary or non-unitary formulations. The unit dose(s) may be contained within a blister pack. The pharmaceutical kit may optionally further comprise instructions for use.

As used herein, the term “pharmaceutical pack” defines an array of one or more unit doses of a pharmaceutical composition, optionally contained within common outer packaging. In pharmaceutical packs comprising a combination of two or more compounds/agents, the individual compounds/agents may unitary or non-unitary formulations. The unit dose(s) may be contained within a blister pack. The pharmaceutical pack may optionally further comprise instructions for use.

As used herein, the term “patient pack” defines a package, prescribed to a patient, which contains pharmaceutical compositions for the whole course of treatment. Patient packs usually contain one or more blister pack(s). Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

The combinations of the invention may produce a therapeutically efficacious effect relative to the therapeutic effect of the individual compounds/agents when administered separately.

The term “ancillary agent” as used herein may define a compound/agent which yields an efficacious combination (as herein defined) when combined with a compound of the formula (1) as defined herein. The ancillary compound may therefore act as an adjunct to the compound of the formula (1) as defined herein, or may otherwise contribute to the efficacy of the combination (for example, by producing a synergistic or additive effect or improving the response rate, as herein defined).

As used herein, the term “antibody” defines whole antibodies (including polyclonal antibodies and monoclonal antibodies (Mabs)). The term is also used herein to refer to antibody fragments, including F(ab), F(ab′), F(ab′)₂, Fv, Fc3 and single chain antibodies (and combinations thereof), which may be produced by recombinant DNA techniques or by enzymatic or chemical cleavage of intact antibodies. The term “antibody” is also used herein to cover bispecific or bifunctional antibodies which are synthetic hybrid antibodies having two different heavy/light chain pairs and two different binding sites. Bispecific antibodies can be produced by a variety of methods including fusion of hybridomas or linking of Fab′ fragments. Also covered by the term “antibody” are chimaeric antibodies (antibodies having a human constant antibody immunoglobulin domain coupled to one or more non-human variable antibody immunoglobulin domain, or fragments thereof). Such chimaeric antibodies therefore include “humanized” antibodies. Also covered by the term “antibody” are minibodies (see WO 94/09817), single chain Fv-Fc fusions and human antibodies antibodies produced by transgenic animals The term “antibody” also includes multimeric antibodies and higher-order complexes of proteins (e.g. heterodimeric antibodies).

### Ancillary Agents for Use According to the Invention

Any of a wide variety of ancillary agents may be used in the combinations of the invention. Preferably, the ancillary agents for use in the combinations of the invention as described herein are selected from the following classes:
1. Antiinflammatory agents;
2. Protease inhibitors;
3. Myostatin antagonists;
4. Cytokines and mobilizing agents;
5. Corticosteroids;
6. Anabolic steroids;
7. TGF-β antagonists;
8. Antioxidants and mitochondrial supporting agents;
9. Dystrophin expression enhancing agents;
10. Gene replacement/repair agents;
11. Cell-based compositions;
12. Creatine;
13. anti-osteoporotic agents;
14. auxiliary utrophin upregulating agents;
15. cGMP signalling modulators; and
16. a combination of two or more of the foregoing classes.

A reference to a particular ancillary agent herein is intended to include ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof).

### 1. Antinflammatory Agents

Muscles affected by DMD show signs of inflammation, including an abundance of macrophages. Thus, a wide range of antiinflammatory agents can be used in the treatment of muscular dystrophies, as discussed below.

### 1.1 Beta2-Adrenergic Receptor Agonists

In one embodiment of the invention, the ancillary agent is a beta2-adrenergic receptor agonist (e.g. albuterol).

Definitions and technical background: The term beta2-adrenergic receptor agonist is used herein to define a class of drugs which act on the β2-adrenergic receptor, thereby causing smooth muscle relaxation resulting in dilation of bronchial passages, vasodilation in muscle and liver, relaxation of uterine muscle and release of insulin. A preferred beta2-adrenergic receptor agonist for use according to the invention is albuterol, an immunosuppressant drug that is widely used in inhalant form for asthmatics. Albuterol is thought to slow disease progression by suppressing the infiltration of macrophages and other immune cells that contribute to inflammatory tissue loss. Albuterol also appears to have some anabolic effects and promotes the growth of muscle tissue. Albuterol may also suppress protein degradation (possibly via calpain inhibition).

1.2 nNOS Stimulators

The loss of dystrophin leads to breaks in the membrane, and destabilizes neuronal nitric oxide synthase (nNOS), a protein which normally generates nitric oxide (NO). It is thought that at least part of the muscle degeneration observed in DMD patients may result from the reduced production of muscle membrane-associated neuronal nitric oxide synthase. This reduction may lead to impaired regulation of the vasoconstrictor response and eventual muscle damage.

### 1.3 Nuclear Factor Kappa-B Inhibitors

A preferred class of antiinflammatory agent suitable for use in the combinations of the invention are Nuclear Factor Kappa-B (NF-kB) inhibitors. NF-kB is a major transcription factor modulating the cellular immune, inflammatory and proliferative responses. NF-kB functions in activated macrophages to promote inflammation and muscle necrosis and in skeletal muscle fibers to limit regeneration through the inhibition of muscle progenitor cells. The activation of this factor in DMD contributes to diseases pathology. Thus, NF-kB plays an important role in the progression of muscular dystrophy and the IKK/NF-B signaling pathway is a potential therapeutic target for the treatment of DMD. Inhibitors of NF-kB (for example, IRFI 042, a vitamin E analogue) ameliorate muscle function, decrease serum CK level and muscle necrosis and enhance muscle regeneration. Furthermore, specific inhibition of NF-kB/IKK-mediated signalling has similar benefits.

### 1.4 TNF-α Antagonists

TNFα is one of the key cytokines that triggers and sustains the inflammation response. In one embodiment of the invention, the ancillary agent is a TNF-α antagonist (e.g. infliximab).

Preferences and specific embodiments: Preferred TNF-α antagonists for use according to the invention include infliximab (Remicade™), a chimeric monoclonal antibody comprising murine VK and VH domains and human constant Fc domains. The drug blocks the action of TNFα by binding to it and preventing it from signaling the receptors for TNFα on the surface of cells. Another preferred TNF-α antagonists for use according to the invention is adalimumab (Humira™). Adalimumab is a fully human monoclonal antibody. Another preferred TNF-α antagonists for use according to the invention is etanercept (Enbrel™). Etanercept is a dimeric fusion protein comprising soluble human TNF receptor linked to an Fc portion of an IgG1. It is a large molecule that binds to and so blocks the action of TNFα. Etanercept mimics the inhibitory effects of naturally occurring soluble TNF receptors, but as a fusion protein it has a greatly extended half-life in the bloodstream and therefore a more profound and long-lasting inhibitory effect. Enbrel is marketed as a lyophylized powder in 25 mg vials which must be reconstituted with a diluent and then injected subcutaneously, typically by the patient at home.

Another preferred TNF-α antagonist for use according to the invention is pentoxifylline (Trental™), chemical name 1-(5-oxohexyl)-3,7-dimethylxanthine. The usual dosage in controlled-release tablet form is one tablet (400 mg) three times a day with meals.

Posology: Remicade is administered by intravenous infusion, typically at 2-month intervals. The recommended dose is 3 mg/kg given as an intravenous infusion followed with additional similar doses at 2 and 6 weeks after the first infusion then every 8 weeks thereafter. For patients who have an incomplete response, consideration may be given to adjusting the dose up to 10 mg/kg or treating as often as every 4 weeks. Humira is marketed in both preloaded 0.8 ml syringes and also in preloaded pen devices, both injected subcutaneously, typically by the patient at home. Etanercept can be administered at a dose of 25 mg (twice weekly) or 50 mg (once weekly).

### 1.5 Ciclosporin

In one embodiment of the invention, the antiinflammatory agent is ciclosporin. Ciclosporin A, the main form of the drug, is a cyclic nonribosomal peptide of 11 amino acids produced by the fungus Tolypocladium inflatum. Ciclosporin is thought to bind to the cytosolic protein cyclophilin (immunophilin) of immunocompetent lymphocytes (especially T-lymphocytes). This complex of ciclosporin and cyclophylin inhibits calcineurin, which under normal circumstances is responsible for activating the transcription of interleukin-2. It also inhibits lymphokine production and interleukin release and therefore leads to a reduced function of effector T-cells. It does not affect cytostatic activity. It has also an effect on mitochondria, preventing the mitochondrial PT pore from opening, thus inhibiting cytochrome c release (a potent apoptotic stimulation factor). Ciclosporin may be administered at a dose of 1-10 mg/kg/day.

### 2. Protease Inhibitors

Proteins in skeletal muscle are degraded by at least three different proteolytic pathways: (a) lysosomal proteases (e.g. the cathepsins); (b) non-lysosomal Ca²⁺-dependent proteases (e.g. calpain); and (c) non-lysosomal ATP-ubiquitin-dependent proteases (e.g. the multicatalytic protease complex or proteasome). Several lines of evidence have suggested that enhanced activation of proteolytic degradation pathways underlies the pathogenesis of muscular dystrophy. Thus, protease inhibitors can be used in the treatment of muscular dystrophies, as discussed below.

Preferred protease inhibitors for use according to the invention may specifically target one of the three degradation pathways described above. Particularly preferred are protease inhibitors which target the non-lysosomal Ca²⁺-dependent pathway (calpain inhibitors) or the non-lysosomal ATP-ubiquitin-dependent pathway (proteasome inhibitors), as described below:

### 2.1 Calpain Inhibitors

In one embodiment of the invention, the ancillary agent is a calpain inhibitor.

Definitions and technical background: The term “calpain inhibitor” is used herein to define any agent capable of inhibiting the activity of calpain. Calpain is a ubiquitous calcium-dependent cysteine protease which cleaves many cytoskeletal and myelin proteins. Calpains belong to a family of Ca²⁺ activated intracellular proteases whose activity is accelerated when abnormal amounts of Ca²⁺ enter the cell by virtue of increased membrane permeability as a result of some traumatic or ischemic event and/or a genetic defect. Calpain is one of a relatively small family of cysteine proteases, which are active in promoting programmed cell death, or apoptosis. It has been implicated in the initiation of both necrotic and apoptotic cell death. When calpain is abnormally up regulated, the accelerated degradation process breaks down cells and tissues faster than they can be restored, resulting in several serious neuromuscular and neurodegenerative diseases. Calpain has been implicated in the accelerated tissue breakdown associated with muscular dystrophies (including DMD). The trigger which activates calpain is Ca²⁺ ions leaking into cells, where the levels are generally very low. The dystrophin gene is involved in maintaining membrane integrity, and when it is mutated, the membrane is more permeable to calcium ions. Thus, the inhibition of calpain activity in the muscles of DMD patients can preserve muscle integrity and prevent or slow muscle deterioration.

Preferences and specific embodiments: Calpain inhibitors for use according to the invention preferably comprise a calpain inhibiting moiety linked to (or associated with) a carrier (which acts to facilitate targeting of the calpain inhibiting moiety to muscle tissue). The targeting moiety may be chemically linked to the calpain inhibiting moiety, or may be physically associated therewith (a liposome carrier). Preferred targeting moieties include carnitine or aminocarnitine. The calpain inhibiting moiety may be leupeptin. Particularly preferred may be Ceptor's Myodur™. Other such calpain inhibitors are described in WO2005124563 (the contents of which are incorporated herein by reference). Other suitable calpain inhibitors are the α-ketocarbonyl calpain inhibitors disclosed in WO 2004/078908 (the contents of which are incorporated herein by reference). Of the calpain inhibitors described in WO 2004/078908, preferred may be those which target both calpain and the proteasome.

The calpain inhibitors for use according to the invention may be chimaeric compounds or combinations in which the calpain inhibiting moiety is associated (e.g. combined with, co-administered with or covalently linked) to a ROS inhibitor. Such agents combine relief of oxidative stress with a reduction in calpain-mediated muscle tissue breakdown. Suitable dual action calpain/ROS inhibitors are described for example in WO01/32654, WO2007/045761, WO2005/056551 and WO 2002/40016 (the contents of which are incorporated herein by reference).

Other suitable calpain inhibitors can be identified using commercially available assay kits (e.g. the calpain activity kit based on a fluorogenic substrate from Oncogene Research Products, San Diego, Calif.). This assay measures the ability of calpain to digest the synthetic substrate Suc-LLVY-AMC: free AMC can be measured fluorometrically at an excitation of 360-380 nm and an emission of 440-460 nm.

### 2.2 Proteasome Inhibitors

Definitions and technical background: Another class of adjunctive agents suitable for use in the combinations of the invention are proteasome inhibitors. Proteasomes control the half-life of many short-lived biological processes. At the plasma membrane of skeletal muscle fibers, dystrophin associates with a multimeric protein complex, termed the dystrophin-glycoprotein complex (DGC). Protein members of this complex are normally absent or greatly reduced in dystrophin-deficient skeletal muscle fibers and inhibition of the proteasomal degradation pathway rescues the expression and subcellular localization of dystrophin-associated proteins. Thus, proteasome inhibitors have recently been identified as potential therapeutics for the treatment of DMD (see Bonuccelli et al. (2003) Am J Pathol. October; 163(4): 1663-1675). The term “proteasome inhibitor” as used herein refers to compounds which directly or indirectly perturb, disrupt, block, modulate or inhibit the action of proteasomes (large protein complexes that are involved in the turnover of other cellular proteins). The term also embraces the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof), as described above.

Preferences and specific embodiments: There are several classes of proteasome Inhibitors suitable for use in the combinations of the invention, including peptide aldehydes (such as MG-132) and the dipeptidyl boronic acid bortezimib (Velcade™; formerly known as PS-341) which is a more specific inhibitor of the proteasome. Thus, preferred proteasome inhibitors for use in accordance with the invention include bortezimib ([(1R)-3-methyl-1-[[(2S)-1-oxo-3-phenyl-2-[(pyrazinylcarbonyl)amino]propyl]amino]butyl]-boronic acid). Bortezimib is commercially available for example from Millennium Pharmaceuticals Inc under the trade name Velcade, or may be prepared for example as described in PCT patent specification No. WO 96/13266, or by processes analogous thereto. Bortezimib specifically interacts with a key amino acid, namely threonine, within the catalytic site of the proteasome. Another preferred proteasome inhibitor for use in the combinations of the invention is the cell-permeable proteasomal inhibitor CBZ-leucyl-leucyl-leucinal (MG-132) (as described in Bonuccelli et al. (2003) Am J Pathol. October; 163(4): 1663-1675, the content of which relating to this compound is incorporated herein by reference). Other inhibitors include those structurally related to MG-132, including MG-115 (CBZ-leucyl-leucyl-norvalinal) and ALLN (N-acetyl-leucyl-leucyl-norleucinal) (as also described in Bonuccelli et al. (2003) Am J Pathol. October; 163(4): 1663-1675, the content of which relating to this compound is incorporated herein by reference).

Posology: The proteasome inhibitor (such as bortezimib) can be administered in a dosage such as 100 to 200 mg/m². These dosages may be administered for example once, twice or more per course of treatment, which may be repeated for example every 7, 14, 21 or 28 days. MG-132 can be administered at a dose of 10 μg/kg/day.

### 3. Myostatin Antagonists

Definitions and technical background: Another class of adjunctive agents suitable for use in the combinations of the invention are myostatin antagonists. Myostatin, also known as growth/differentiation factor 8 (GDF-8) is a transforming growth factor-β (TGF-β) family member involved in the regulation of skeletal muscle mass. Most members of the TGF-β-GDF family are widely expressed and are pleiotropic: however, myostatin is primarily expressed in skeletal muscle tissue where it negatively controls skeletal muscle growth. Myostatin is synthesized as an inactive preproprotein which is activated by proteolyic cleavage. The precurser protein is cleaved to produce an approximately 109 amino acid COOH-terminal protein which, in the form of a homodimer of about 25 kDa, is the mature, active form. The mature dimer appears to circulate in the blood as an inactive latent complex bound to the propeptide. As used herein the term “myostatin antagonist” defines a class of agents which inhibit or block at least one activity of myostatin, or alternatively, blocks or reduces the expression of myostatin or its receptor (for example, by interference with the binding of myostatin to its receptor and/or blocking signal transduction resulting from the binding of myostatin to its receptor). Such agents therefore include agents which bind to myostatin itself or to its receptor.

Preferences and specific embodiments: Myostatin antagonists for use according to the invention include antibodies to GDF-8; antibodies to GDF-8 receptors; soluble GDF-8 receptors and fragments thereof (e.g. the ActRIIB fusion polypeptides as described in U.S. Ser. No. 10/689,677, including soluble ActRIIB receptors in which ActRIIB is joined to the Fc portion of an immunoglobulin); GDF-8 propeptide and modified forms thereof (e.g. as described in WO 02/068650 or U.S. Ser. No. 10/071,499, including forms in which GDF-8 propeptide is joined to the Fc portion of an immunoglobulin and/or form in which GDF-8 is mutated at an aspartate (asp) residue, e.g., asp-99 in murine GDF-8 propeptide and asp-100 in human GDF-8 propeptide); a small molecule inhibitor of GDF-8; follistatin (e.g. as described in U.S. Pat. No. 6,004,937) or follistatin-domain-containing proteins (e.g. GASP-1 or other proteins as described in U.S. Ser. No. 10/369,736 and U.S. Ser. No. 10/369,738); and modulators of metalloprotease activity that affect GDF-8 activation, as described in U.S. Ser. No. 10/662,438.

Preferred myostatin antagonists include myostatin antibodies which bind to and inhibit or neutralize myostatin (including the myostatin proprotein and/or mature protein, in monomeric or dimeric form). Myostatin antibodies are preferably mammalian or non-mammalian derived antibodies, for example an IgNAR antibody derived from sharks, or humanised antibodies (or comprise a functional fragment derived from antibodie. Such antibodies are described, for example, in US 2004/0142383, US 2003/1038422, WO 2005/094446 and WO 2006/116269 (the content of which is incorporated herein by reference). Myostatin antibodies also include those which bind to the myostatin proprotein and prevent cleavage into the mature active form. A particularly preferred myostatin antibody for use in the combinations of the invention is Wyeth's Stamulumab (MYO-029). MYO-029 is a recombinant human antibody which binds to and inhibits the activity of myostatin. Other preferred antibody antagonists include the antibodies described in U.S. Pat. No. 6,096,506 and U.S. Pat. No. 6,468,535 (incorporated herein by reference). In some embodiments, the GDF-8 inhibitor is a monoclonal antibody or a fragment thereof that blocks GDF-8 binding to its receptor. Other illustrative embodiments include murine monoclonal antibody JA-16 (as described in US2003/0138422 (ATCC Deposit No. PTA-4236); humanized derivatives thereof and fully human monoclonal anti-GDF-8 antibodies (e.g., Myo-29, Myo-28 and Myo-22, ATCC Deposit Nos. PTA-4741, PTA-4740, and PTA-4739, respectively, or derivatives thereof) as described in US2004/0142382 and incorporated herein by reference.

Other preferred myostatin antagonists include soluble receptors which bind to myostatin and inhibit at least one activity thereof. The term “soluble receptor” here includes truncated versions or fragments of the myostatin receptor which specifically bind myostatin thereby blocking or inhibiting myostatin signal transduction. Truncated versions of the myostatin receptor, for example, include the naturally-occurring soluble domains, as well as variations elaborated by proteolysis of the N- or C-termini. The soluble domain includes all or part of the extracellular domain of the receptor, either alone or attached to additional peptides or other moieties. Since myostatin binds activin receptors (including activin type IEB receptor (ActRHB) and activin type HA receptor (ActRHA), activin receptors can form the basis of soluble receptor antagonists. Soluble receptor fusion proteins can also be used, including soluble receptor Fc (see US2004/0223966 and WO2006/012627, both of which are incorporated herein by reference).

Other preferred myostatin antagonists based on the myostatin receptors are ALK-5 and/or ALK-7 inhibitors (see for example WO2006025988 and WO2005084699, the disclosure of which is incorporated herein by reference). As a TGF-β cytokine, myostatin signals through a family of single transmembrane serine/threonine kinase receptors. These receptors can be divided in two classes, the type I or activin like kinase (ALK) receptors and type II receptors. The ALK receptors are distinguished from the Type II receptors in that the ALK receptors (a) lack the serine/threonine rich intracellular tail, (b) possess serine/threonine kinase domains that are very homologous between Type I receptors, and (c) share a common sequence motif called the GS domain, consisting of a region rich in glycine and serine residues. The GS domain is at the amino terminal end of the intracellular kinase domain and is believed to be critical for activation by the Type II receptor. Several studies have shown that TGF-β signaling requires both the ALK (Type I) and Type II receptors. Specifically, the Type II receptor phosphorylates the GS domain of the Type I receptor for TGF-[beta] ALK5, in the presence of TGF-[beta]. The ALK5, in turn, phosphorylates the cytoplasmic proteins smad2 and smad3 at two carboxy terminal serines. Generally, it is believed that in many species, the Type II receptors regulate cell proliferation and the Type I receptors regulate matrix production. Various ALK5 receptor inhibitors have been described (see, for example, U.S. Pat. No. 6,465,493, US2003/0149277, US2003/0166633, US20040063745, and US2004/0039198, the disclosure of which is incorporated herein by reference). Thus, the myostatin antagonists for use according to the invention may comprise the myostatin binding domain of an ALK5 and/or ALK7 receptor.

Other preferred myostatin antagonists include soluble ligand antagonists which compete with myostatin for binding to myostatin receptors. The term “soluble ligand antagonist” here refers to soluble peptides, polypeptides or peptidomimetics capable of non-productively binding the myostatin receptor(s) (e.g. the activin type HB receptor (ActRHA)) and thereby competitively blocking myostatin-receptor signal transduction. Soluble ligand antagonists include variants of myostatin, also referred to as “myostatin analogues” that have homology with but not the activity of myostatin. Such analogues include truncates (such an N- or C-terminal truncations, substitutions, deletions, and other alterations in the amino acid sequence, such as variants having non-amino acid substitutions).

Other preferred myostatin antagonists further include polynucleotide antagonists. These antagonists include antisense or sense oligonucleotides comprising a single-stranded polynucleotide sequence (either RNA or DNA) capable of binding to target mRNA (sense) or DNA (antisense) sequences. Antisense or sense oligonucleotides for use according to the invention comprise fragments of the targeted polynucleotide sequence encoding myostatin or its receptor, transcription factors, or other polynucleotides involved in the expression of myostatin or its receptor. Such a fragment generally comprises at least about 14 nucleotides, typically from about 14 to about 30 nucleotides. Antisense or sense oligonucleotides further comprise oligonucleotides having modified sugar-phosphodiester backbones (or other sugar linkages, such as those described in WO 91/06629) and wherein such sugar linkages are resistant to endogenous nucleases. Such oligonucleotides with resistant sugar linkages are stable in vivo but retain sequence specificity to be able to bind to target nucleotide sequences. Other examples of sense or antisense oligonucleotides include those oligonucleotides which are covalently linked to organic moieties, such as those described in WO 90/10448, and other moieties that increases affinity of the oligonucleotide for a target nucleic acid sequence, such as poly-(L)-lysine and morpholinos. Further still, intercalating agents, such as ellipticine, and alkylating agents or metal complexes may be attached to sense or antisense oligonucleotides to modify binding specificities of the antisense or sense oligonucleotide for the target nucleotide sequence. Thus, RNA interference (RNAi) produced by the introduction of specific small interfering RNA (siRNA), may also be used to inhibit or eliminate the activity of myostatin.

Particularly preferred myostatin antagonists include but are not limited to follistatin, the myostatin prodomain, growth and differentiation factor 11 (GDF-11) prodomain, prodomain fusion proteins, antagonistic antibodies that bind to myostatin, antagonistic antibodies or antibody fragments that bind to the activin type IEB receptor, soluble activin type IHB receptor, soluble activin type IEB receptor fusion proteins, soluble myostatin analogs (soluble ligands), oligonucleotides, small molecules, peptidomimetics, and myostatin binding agents disclose anti-myostatin antibodies. Other preferred antagonists include the peptide immunogens described in U.S. Pat. No. 6,369,201 and WO 01/05820 (incorporated herein by reference) and myostatin multimers and immunoconjugates capable of eliciting an immune response and thereby blocking myostatin activity. Other preferred antagonists include the protein inhibitors of myostatin described in WO02/085306 (and incorporated herein by reference), which include the truncated Activin type II receptor, the myostatin pro-domain, and follistatin. Other myostatin inhibitors include those released into culture from cells overexpressing myostatin (see WO00/43781), dominant negatives of myostatin (see WO 01/53350) including the Piedmontese allele, and mature myostatin peptides having a C-terminal truncation at a position either at or between amino acid positions 335 to 375. The small peptides described in US2004/0181033 (incorporated herein by reference) which comprise the amino acid sequence WMCPP, are also suitable for use in the combinations of the invention.

### 4. Cytokines and Mobilizing Agents

Definitions and technical background: Another class of adjunctive agents suitable for use in the combinations of the invention are cytokines, and in particular anabolic cytokines and insulin-like growth factors (such as IGF-1 or IGF-2). The anabolic effect of IGF-1 on muscle is very well established. In muscular dystrophies, a progressive reduction in the proliferative capacity of satellite cells occurs and this loss of proliferative capacity may be ameliorated by treatment with IGF-1. Thus, IGF-1 (and other members of this class of cytokine) may help to slow the progress of the dystrophinopathies by enhancing activation of dormant satellite cells. Insulin-like growth factors (IGFs) are members of the highly diverse insulin gene family that includes insulin, IGF-I, IGF-II, relaxin, prothoraciotropic hormone (PTTH), and molluscan insulin-related peptide. The IGFs are circulating, mitogenic peptide hormones that have an important role in stimulating growth, differentiation, metabolism and regeneration both in vitro and in vivo.

Preferences and specific embodiments: Preferred cytokines for use according to the invention include IGF-1 and IGF-2. Approximately 99% of IGF-1 in healthy individuals circulates in the blood stream bound to IGFBP-3 where it forms a large ternary 150 kD complex after association with acid-labile subunit protein (ALS). The ternary complex is restricted to the circulation by the capillary endothelium and thus serves as a circulatory reservoir of IGF-1. Thus, for therapeutic applications according to the invention IGF-1 is preferably administered in the form of a complex. For example, a preferred cytokine for use in the combinations of the invention is IPLEX™ (recombinant protein complex of insulin-like growth factor-I (IGF-1) and its most abundant binding protein, insulin-like growth factor binding protein-3 (IGFBP-3)). Another suitable cytokine is G-CSF (or other mobilizing agents as herein defined, e.g. GM-CSF), which can support muscle regeneration by mobilizing stem cells from the marrow. Other preferred cytokines include IGF-1 derivatives (IGF-1 E peptides) as described in WO2006056885 (the content of which is incorporated herein by reference) which have the appropriate subsets of the function of the full-length IGF-1 and, in particular, its regenerative capacity. Thus, in a preferred embodiment the combinations of the invention comprise the IGF-I Ea peptide (i.e. the 35 amino acid C terminal peptide translated from part of exons 4 and 5 of the IGF-I gene as part of the IGF-I propeptide and which is cleaved off during post-translational processing) and/or the IGF-I Eb peptide (i.e. the 41 amino acid C terminal peptide translated from parts of exons 4, 5 and 6 of the IGF-I gene as part of the IGF-I propeptide and which is cleaved off during post-translational processing).

Posology: IPLEX™ can be administered via subcutaneous injection at an initial dose of 0.5 mg/kg, to be increased into the therapeutic dose range of 1 to 2 mg/kg, given once daily. IPLEX™ can be given in the morning or in the evening but should be administered at approximately the same time every day. In order to establish tolerability to IPLEX™, glucose monitoring should be considered at treatment initiation or when a dose has been increased. If frequent symptoms of hypoglycemia or severe hypoglycemia occur, preprandial glucose monitoring should continue. Glucose monitoring is also advised for patients with recent occurrences of asymptomatic or symptomatic hypoglycemia. If evidence of hypoglycemia is present at the time of dosing, the dose should be withheld.

Dosage can be titrated up to a maximum of 2 mg/kg daily based on measurement of IGF-1 levels obtained 8-18 hours after the previous dose. Dosage should be adjusted downward in the event of adverse effects (including hypoglycemia) and/or IGF-1 levels that are greater than or equal to 3 standard deviations above the normal reference range for IGF-1.

### 5. Corticosteroids

In one embodiment of the invention, the ancillary agent is a corticosteroid.

Definition and biological activities: The term “corticosteroid” as used herein refers to any of several steroid hormones secreted by the cortex of the adrenal glands and which are involved in one or more of the following physiological processes: stress response, immune response and regulation of inflammation, carbohydrate metabolism, protein catabolism and blood electrolyte levels. The term also includes synthetic analogues which share the aforementioned properties. Corticosteroids include glucocorticoids and mineralocorticoids. Glucocorticoids control carbohydrate, fat and protein metabolism and are anti-inflammatory. Mineralocorticoids control electrolyte and water levels, mainly by promoting sodium retention in the kidney. Some corticosteroids have dual glucocorticoid and mineralocorticoid activities. For example, prednisone (see below) and its derivatives have some mineralocorticoid action in addition to a glucocorticoid effect. The precise cellular mechanism(s) by which corticosteroids produce antidystrophic effects are not yet known. A multifactorial mechanism is likely and the effects of corticosteroids probably involve a reduction of inflammation, suppression of the immune system, improvement in calcium homeostasis, upregulation of the expression of compensatory proteins and an increase in myoblast proliferation.

Problems: The use of corticosteroids is associated with side effects which vary from person to person and on the dosage of the regime used, but they can be severe. The most common side effects are weight gain and mood changes. Weight gain (and attendant changes in muscle activity and use) can abrogate some of the benefits of treatment. Long-term use may lead to growth suppression, cataracts, osteoporosis and muscle atrophy (affecting the same proximal muscles affected in DMD and BMD). These side effects may limit the long-term effectiveness of corticosteroid therapy. Other side effects include hypertension, diabetes, skin atrophy, poor wound healing and immunosuppression. Deflazacort was evaluated in the hope that it would have fewer side effects than prednisone.

Preferences and Specific embodiments: Preferred are glucocorticoids (or corticosteroids having dual glucocorticoid/minerlocorticoid activity). Synthetic corticosteroids are preferred. In one embodiment, the corticosteroid is prednisone (prodrug) or prednisolone (liver metabolite of prednisone and active drug). In another embodiment, the corticosteroid is deflazacort. Deflazacort is an oxazoline analogue of prednisone. Other synthetic corticosteroids suitable for use in the combinations of the invention include one or more corticosteroids selected from: alclometasone, amcinonide, beclomethasone (including beclomethasone dipropionate), betamethasone, budesonide, ciclesonide, clobetasol, clobetasone, clocortolone, cloprednol, cortivazol, deoxycorticosterone, desonide, desoximetasone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, halcinonide, halometasone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednylidene, rimexolone, tixocortol, triamcinolone and ulobetasol (or combinations and/or derivatives (e.g. pharmaceutically acceptable salts) of one or more of the foregoing). Suitable endogenous corticosteroids for use in the combinations of the invention include include one or more corticosteroids selected from aldosterone, cortisone, hydrocortisone/cortisol and desoxycortone (or combinations and/or derivatives (e.g. pharmaceutically acceptable salts) of one or more of the foregoing).

Posology. Prednisone may be administered daily in dosages ranging from 0.3 to 1.5 mg/kg (typically 0.7 mg/kg). Some patients respond better to ≧2.5 mg/kg every other day. Deflazacort has an estimated dosage equivalency of 1:1.3 compared with prednisone, though biological equivalence between deflazacort and prednisone also depends on the specific actions under examination. Corticosteroids (including delazacort and prednisone) are usually taken orally but can be delivered by intramuscular injection.

### 6. Anabolic Steroids

In one embodiment of the invention, the ancillary agent is an anabolic steroid.

Definition and biological activities: The term “anabolic steroid” as used herein refers to any of several steroid hormones related to the male hormone testosterone and synthetic analogues thereof. Such steroids are may also be referred to as “anabolic-androgenic steroids” or “AAS”. Anabolic steroids increase protein synthesis within cells, promoting anabolism (especially in muscles). The precise cellular mechanism(s) by which anabolic steroids produce antidystrophic effects are not yet known, but it seems that their anabolic effects in muscles effectively compensates for muscle loss. Oxandrolone has been shown to have anabolic effects on DMD muscle as well as decreasing muscle degeneration and so easing the demands for muscle regeneration. By conserving regenerative capacity, anabolic steroids such as oxandrolone may prolong muscle function.

Problems: The use of anabolic steroids is associated with severe side effects. The most common side effects are liver and kidney damage, sterility, stunting of growth and severe mood swings. Anabolic steroids also also tend to be androgenizing and can promote growth of beard and body hair, maturation of genitalia and development of acne. Withdrawal can lead to rapid and severe deterioration in muscle mass and function.

Preferences and Specific embodiments: Preferred are synthetic anabolic steroids such as oxandrolone (Anavar), norethandrolone and methandrostenolone (Dianabol). Oxandrolone (an oral synthetic analog of testosterone) may be particularly preferred because in addition to its anabolic properties it also blocks the binding of cortisol to glucocorticoid receptors on muscle, thus preventing muscle breakdown. Other anabolic steroids suitable for use in the combinations of the invention include one or more anabolic steroids selected from: DHEA, DHT, methenolone, oxymetholone, quinbolone, stanozolol, ethylestrenol, nandrolone (Deca Durabolin), oxabolone cipionate, boldenone undecylenate (Equipoise), stanozolol (Winstrol), oxymetholone (Anadrol-50), fluoxymesterone (Halotestin), trenbolone (Fina), methenolone enanthate (Primobolan), 4-chlordehydromethyltestosterone (Turinabol), mesterolone (Proviron), mibolerone (Cheque Drops), tetrahydrogestrinone and testosterone (or combinations and/or derivatives (e.g. pharmaceutically acceptable salts) of one or more of the foregoing).

Posology: Anabolic steroids may be administered as orally in the form of pills, by injection or via skin patches. Oral administration is most convenient, but since the steroid must be chemically modified so that the liver cannot break it down before it reaches the blood stream these formulations can cause liver damage in high doses. Injectable steroids are typically administered intramuscularly. Transdermal patches can be sued to deliver a steady dose through the skin and into the bloodstream. Oxandrolone may be administered orally at a daily dosage of 0.1 mg/kg.

### 7. TGF-β Antagonists

Definitions and technical background: Transforming growth factor beta (TGF-β) promotes fibrosis in response to muscle tissue damage associated with DMD that can contribute to disease pathology. In one embodiment of the invention, the ancillary agent is a TGF-β antagonist.

The term TGF-β antagonist is used herein to refer to compounds which directly or indirectly perturb, disrupt, block, modulate or inhibit the action of TGF-β. The term also embraces the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof).

Preferences and specific embodiments: Preferred TGF-β antagonists for use according to the invention include anti-TGF-β antibodies, tamoxifen, losartan and pirfenidone. Pirfenodone is an orally active synthetic antifibrotic agent structurally similar to pyridine 2,4-dicarboxylate. Pirfenidone inhibits fibroblast, epidermal, platelet-derived, and TGF-β-1 growth factors and also inhibits DNA synthesis and the production of mRNA for collagen types I and III, resulting in a reduction in radiation-induced fibrosis. Losartan is an angiotensin II receptor antagonist drug used mainly to treat hypertension currently marketed by Merck & Co. under the trade name Cozaar™. However, losartan also downregulates the expression of (TGF-β types I and II receptors. Tamoxifen is an orally active selective estrogen receptor modulator (SERM) which is used in the treatment of breast cancer and is currently the world's largest selling drug for this indication. Tamoxifen is sold under the trade names Nolvadex™, Istubal™ and Valodex™. Tamoxifen may be administered at a dose of 10-100 mg per day (e.g. 20-40 mg/day).

### 8. Antioxidants and Mitochondrial Supporting Agents

In Duchenne Muscular Dystrophy (DMD), the cytoskeletal protein dystrophin is absent leading to numerous cellular dysfunctions that culminate in muscle cell necrosis. Subsequently, an inflammatory response develops in the necrotic muscle tissue, resulting in increased oxidative stress, responsible for further tissue damage. In the mdx dystrophic mouse, both inflammation and oxidative stress have been identified as aggravating factors for the course of the disease.

GTE and EGCG also display unexpected pro myogenic properties. Primary cultures of skeletal muscle cells were established from both normal and dystrophic mice and treated with GTE and EGCG for 1-7 days. As judged by in situ staining of myosin heavy chains (MyHC), we found that GTE and EGCG concentration-dependently stimulated the rate of formation of myotubes within the first 2-4 days of application. The amount of myotubes reached similar level with both agents compared to control thereafter. Western-blot analysis was performed on myotube cultures treated for 7 days. GTE and EGCG promoted the expression of several muscle-specific proteins, such as dystrophin (in control cultures), sarcomeric alpha actinin, and MyHC, while myogenin was unchanged. By contrast, the expression of desmin was down-regulated and redistributed to Z discs. Our results suggest that green tea polyphenols display pro myogenic properties by acting directly on skeletal muscle cells. These findings suggest a beneficial action for muscle regeneration and strengthening in dystrophic condition.

Green tea polyphenols, such as epigallocatechin gallate (EGCG), are known to be powerful antioxidants. Because inflammation is involved in the degradation of muscle tissue in MD, oxidative stress is believed to play a role in this process. Thus, green tea and its active constituents (including EGCG and other polyphenols) may improve MD prognosis by reducing this oxidative stress. Feeding studies with mdx mice have shown a protective effect of EGCG against the first massive wave of necrosis. It also stimulated muscle adaptation toward a stronger and more resistant phenotype. The effective dosage corresponds to about seven cups of brewed green tea per day in humans

Coenzyme Q10 (CoQ10; also called ubiquitin) is a powerful antioxidant and mitochondrial respiratory chain cofactor. It possesses membrane-stabilizing properties and is capable of penetrating cell membranes and mitochondria. Dosages of 100 mg CoQ10 daily for three months have been shown to be beneficial in human trials, though higher dosages are likely to yield better results.

Idebenone is a synthetic analog of Coenzyme Q10 and is thought to perform the same functions as CoQ10 without the risk of auto-oxidation. Like CoQ10, idebenone can therefore contribute to maintaining correct electron balance, which is necessary for the production of cellular energy. Since muscle cells are particularly energy-demanding idebenone and CoQ10 can preserve mitochondrial function and protect cells from oxidative stress.

Glutamine is an important energy source and acute oral glutamine administration appears to have a protein-sparing effect. Arginine (and other pharmacological activators of the NO pathway) may enhance the production of utrophin in MDX mice. The increase is likely to be mediated by arginine-fueled production of nitric oxide (NO), which plays an important role in blood vessel function and is generally lower in people with MD. Studies with MDX mice have also shown that a combination of arginine and deflazacort may be more beneficial than deflazacort alone.

Other antioxidants suitable for use according to the invention are the chimaeric compounds or combinations in which the a ROS inhibitor is associated (e.g. combined with, co-administered with or covalently linked) to calpain inhibiting moiety. Such agents combine relief of oxidative stress with a reduction in calpain-mediated muscle tissue breakdown. Suitable dual action calpain/ROS inhibitors are described for example in WO01/32654, WO2007/045761, WO2005/056551 and WO 2002/40016 (the contents of which are incorporated herein by reference).

### 9. Dystrophin Expression Enhancing Agents

### 9.1 Read-Through Agents

A subset of DMD patients (around 15%) have a nonsense mutation that produces a premature stop signal in their RNA, resulting in abnormal truncation of protein translation. In one embodiment of the invention, the ancillary agent is an agent which promotes readthrough of premature stop codons (“read-through agent”), thereby bypassing the premature stop codon and restoring the expression of full-length, functional dystrophin.

Suitable read-through agents for use according to the invention are 1,2,4-oxadiazole compounds as described in U.S. Pat. No. 6,992,096 (which is incorporated herein by reference):

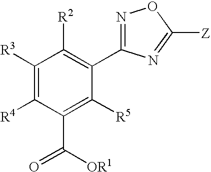

One such compound is 3-[5-(2-fluoro-phenyl)-[I,2,4]oxadiazol-3-yl]-benzoic acid. A preferred readthrough agent is PTC124. PTC124 is a 284-Dalton 1,2,4-oxadiazole that promotes ribosomal readthrough of premature stop codons in mRNA. Thus, the combinations of the invention may comprise 1,2,4-oxadiazole benzoic acid compounds (including 3-[5-(2-fluoro-phenyl)-[I,2,4]oxadiazol-3-yl]-benzoic acid) (see e.g. WO2006110483, the content of which is incorporated herein by reference).

PTC124, 3-[5-(2-fluoro-phenyl)-[I,2,4]oxadiazol-3-yl]-benzoic acid or a pharmaceutically acceptable salt, solvate or hydrate thereof can be administered in single or divided (e.g., three times daily) doses between 0.1 mg/kg and 500 mg/kg, 1 mg/kg and 250 mg/kg, 1 mg/kg and 150 mg/kg, 1 mg/kg and 100 mg/kg, 1 mg/kg and 50 mg/kg, 1 mg/kg and 25 mg/kg, 1 mg/kg and 10 mg/kg or 2 mg/kg and 10 mg/kg to a patent in need thereof. In a particular embodiment, the 3-[5-(2-fluoro-phenyl)-[I,2,4]oxadiazol-3-yl]-benzoic acid or a pharmaceutically acceptable salt, solvate or hydrate thereof is administered in a dose of about 4 mg/kg, about 7 mg/kg, about 8 mg/kg, about 10 mg/kg, about 14 mg/kg or about 20 mg/kg.

Other readthrough agents for use according to the invention include aminoglycoside antibiotics, including gentamicin. Particularly preferred may be aminoglycosides that contain a 6′ hydroxyl group (e.g. paromomycin), which may be effective at lower doses and may display less toxicity than compounds such as gentamicin.

### 9.2 Exon Skipping

Most cases of Duchenne muscular dystrophy (DMD) are caused by dystrophin gene mutations that disrupt the mRNA reading frame. In some cases, forced exclusion (skipping) of a single exon can restore the reading frame, giving rise to a shorter, but still functional dystrophin protein (so called quasi-dystrophin). Antisense oligonucleotides (AONs) designed to cause exon skipping can target a broader range of mutations than can compounds that cause cells to ignore premature stop codons by induce cells to leave out sections of genetic instructions that contain mistakes and join together the surrounding, correct instructions. However, since AONs are not self-renewed, they cannot achieve long-term correction. To overcome this limitation, antisense sequences can be introduced into small nuclear RNAs (snRNA) and vectorized in AAV and lentiviral vectors.

### 10. Gene Replacement/Repair Agents

In one embodiment of the invention, the ancillary agent is a nucleic acid construct adapted to replace or repair non-functional endogenous genetic material. Gene therapy may be adeno-associated virus (AAV) vector-mediated gene therapy, preferably using the microdystrophin gene. Highly abbreviated microdystrophin cDNAs have been developed for adeno-associated virus (AAV)-mediated DMD gene therapy. Among these, a C-terminal-truncated ΔR4-R23/ΔC microgene (AR4/AC) is a very promising therapeutic candidate gene.

Targeted correction of mutations in the genome holds great promise for the repair/treatment of disease causing mutations either on their own applied directly to the affected tissue, or in combination with other techniques such as stem cell transplantation. Various DNA or RNA/DNA based Corrective Nucleic Acid (CNA) molecules such as chimeraplasts, single stranded oligonucleotides, triplex forming oligonucleotides and SFHR have been used to change specific mutant loci. MyoDys® is comprised of plasmid DNA encoding the full-length human dystrophin gene. Mirus' Pathway IV™ delivery technology is used to administer the pDNA to a patient's limb skeletal muscles.

### 11. Cell-Based Therapies

In one embodiment of the invention, the ancillary agent is a myogenic cell or tissue composition. Various types of myogenic cell have been shown to have potential in the treatment of DMD, including stem cells from umbilical cord, mesenchymal stem cells and muscle-derived stem cells.

### 12. Creatine

Definition and biological activities: Creatine is an energy precursor that is naturally produced by the body. Creatine kinase (CK) phosphorylates creatine for later donation to contractile muscle filaments: phosphocreatine enters muscle cells and promotes protein synthesis while reducing protein breakdown. In healthy individuals, creatine has been shown to enhance endurance and increase energy levels by preventing depletion of adenosine triphosphate. Among MD patients, studies have suggested that supplemental creatine can improve muscle performance and strength, decrease fatigue, and slightly improve bone mineral density.

Problems: High doses of creatine can cause kidney damage and requires cohydration. Behavioral changes have been recorded.

Posology: Creatine can be administered as a powdered nutritional supplement. In recent trials with DMD patients, slight increases in muscle strength on administration of low levels (1 to 10 g/day) of creatine monohydrate have been recorded. Intermittent administration (involving a break of one to several weeks) may mitigate side effects whilst providing the same benefits as constant use. Dosages in the region of 100 mg/kg/day are well-tolerated and have been found to decrease bone degradation and increase strength and fat-free mass. Benefits have been reported for the co-administration of creatine with conjugated linoleic acid (alpha-lipoic acid), hydroxyl-beta-methylbutyrate and prednisolone.

### 13. Anti-Osteoporotic Agents

Combined therapy to inhibit bone resorption, prevent osteoporosis, reduce skeletal fracture, enhance the healing of bone fractures, stimulate bone formation and increase bone mineral density can be effectuated by combinations comprising various anti-osteoporotic agents. Preferred are bisphosphonates including alendronate, tiludronate, dimethyl-APD, risedronate, etidronate, YM-175, clodronate, pamidronate and BM-210995 (ibandronate). Others include oestrogen agonist/antagonists. The term oestrogen agonist/antagonists refers to compounds which bind with the estrogen receptor, inhibit bone turnover and prevent bone loss. In particular, oestrogen agonists are herein defined as chemical compounds capable of binding to the estrogen receptor sites in mammalian tissue, and mimicking the actions of estrogen in one or more tissue. Exemplary oestrogen agonist/antagonists include droloxifene and associated compounds (see U.S. Pat. No. 5,047,431), tamoxifen and associated compounds (see U.S. Pat. No. 4,536,516), 4-hydroxy tamoxifen (see U.S. Pat. No. 4,623,660), raloxifene and associated compounds (see U.S. Pat. No. 4,418,068 and idoxifene and associated compounds (see U.S. Pat. No. 4,839,155).

### 14. Auxiliary Utrophin Upregulating Agents

In addition to the compounds of formula (I) as defined herein, the combinations of the present invention may include one or more auxiliary utrophin upregulating agents. Such auxiliary utrophin upregulating agents are compounds that upregulate (i.e. increase the expression or activity of utrophin) and which do not conform to the structure of formula (I) as defined herein (or the ionic, salt, solvate, isomers, tautomers, N-oxides, ester, prodrugs, isotopes and protected forms thereof). The auxiliary utrophin upregulating agents for use in the combinations of the invention preferably upregulate utrophin via a mechanism that is different from that of the compounds of formula (I) and (II) described herein.

15. cGMP Signalling Modulators

It has recently been shown (Khairallah et al., (2008) PNAS 105(19): 7028-7033) that enhancement of cGMP signaling by administration of the phosphodiesterase 5 (PDE5) inhibitor sildenafil prevents deterioration of myocardial contractile performance in mdx hearts.

Thus, cGMP signaling enhancers, including in particular selective PDE5 inhibitors (including for example sildenafil, tadalafil, vardenafil, udenafil and avanafil) may be used in combination with the compounds of the invention to treat DMD or BMD. Such combinations find particular application in the treatment of dystrophic cardiomyopathies and may be used to prevent or delay the onset of dystrophin-related cardiomyopathies as the clinical course of DMD/BMD progresses.

Thus, the invention contemplates combinations of the compounds of the invention with cGMP signaling enhancers, including in particular selective PDE5 inhibitors. Preferred combinations are comprise a compound of the invention and a PDE5 inhibitor selected from sildenafil, tadalafil, vardenafil, udenafil and avanafil. Particularly preferred is a combination comprising a compound of the invention and sildenafil. The compound of the invention for use in the aforementioned combinations is preferably compound number 390 of Table 1 being 5-(ethylsulfonyl)-2-(naphthalen-2-yl)benzo[d]oxazole.

### Formulation and Posology

The compounds of formula I for use in the treatment of DMD will generally be administered in the form of a pharmaceutical composition.

Thus, according to a further aspect of the invention there is provided a pharmaceutical composition including preferably less than 80% w/w, more preferably less than 50% w/w, e.g. 0.1 to 20%, of a compound of formula I, or a pharmaceutically acceptable salt thereof, as defined above, in admixture with a pharmaceutically acceptable diluent or carrier.

We also provide a process for the production of such a pharmaceutical composition which comprises mixing the ingredients. Examples of pharmaceutical formulations which may be used, and suitable diluents or carriers, are as follows:
for intravenous injection or infusion—purified water or saline solution;
for inhalation compositions—coarse lactose;
for tablets, capsules and dragees—microcrystalline cellulose, calcium phosphate, diatomaceous earth, a sugar such as lactose, dextrose or mannitol, talc, stearic acid, starch, sodium bicarbonate and/or gelatin;
for suppositories—natural or hardened oils or waxes.

When the compound is to be used in aqueous solution, e.g. for infusion, it may be necessary to incorporate other excipients. In particular there may be mentioned chelating or sequestering agents, antioxidants, tonicity adjusting agents, pH-modifying agents and buffering agents.

Solutions containing a compound of formula I may, if desired, be evaporated, e.g. by freeze drying or spray drying, to give a solid composition, which may be reconstituted prior to use.

When not in solution, the compound of formula I preferably is in a form having a mass median diameter of from 0.01 to 10 μm. The compositions may also contain suitable preserving, stabilising and wetting agents, solubilisers, e.g. a water-soluble cellulose polymer such as hydroxypropyl methylcellulose, or a water-soluble glycol such as propylene glycol, sweetening and colouring agents and flavourings. Where appropriate, the compositions may be formulated in sustained release form.

The content of compound formula I in a pharmaceutical composition is generally about 0.01-about 99.9 wt %, preferably about 0.1-about 50 wt %, relative to the entire preparation.

The dose of the compound of formula I is determined in consideration of age, body weight, general health condition, diet, administration time, administration method, clearance rate, combination of drugs, the level of disease for which the patient is under treatment then, and other factors.

While the dose varies depending on the target disease, condition, subject of administration, administration method and the like, for oral administration as a therapeutic agent for the treatment of Duchenne muscular dystrophy in a patient suffering from such a disease is from 0.01 mg-10 g, preferably 0.1-100 mg, is preferably administered in a single dose or in 2 or 3 portions per day.

### EXAMPLES

The potential activity of the compounds of formula I for use in the treatment of DMD may be demonstrated in the following predictive assay and screens.

### 1. Luciferase Reporter Assay (Murine H2K Cells)

The cell line used for the screen is an immortalized mdx mouse H2K cell line that has been stably transfected with a plasmid containing ≈5 kb fragment of the Utrophin A promoter including the first untranslated axon linked to a luciferase reporter gene.

Under conditions of low temperature and interferon containing media, the cells remain as myoblasts. These are plated into 96 well plates and cultured in the presence of compound for three days. The level of luciferase is then determined by cell lysis and reading of the light output from the expressed luciferase gene utilising a plate luminometer.

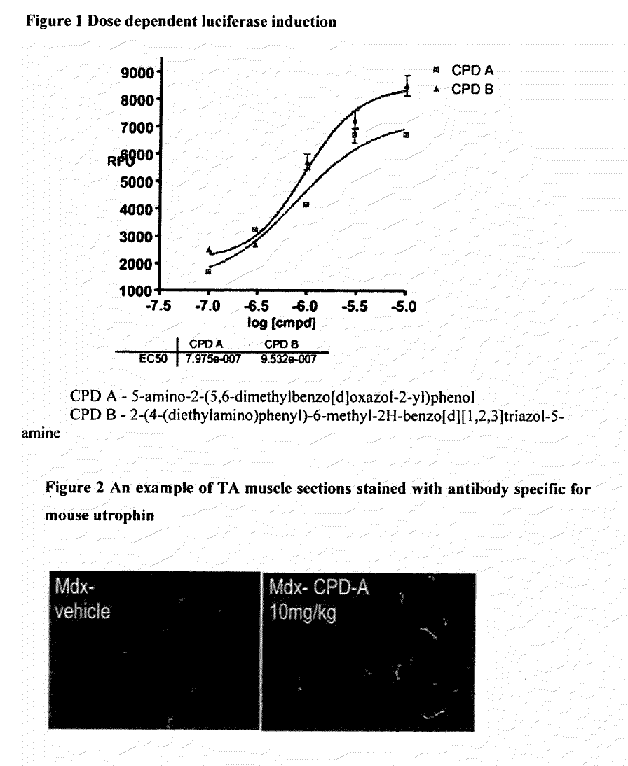

Example of pharmacological dose response of compounds in the assay is shown in FIG. 1

### 2. mdx Mouse

Data obtained from the ADMET data was prioritised and the compounds with the best in vitro luciferase activity and reasonable ADMET data were prioritised for testing in the mdx proof of concept study where the outcome was to identify whether any of the compounds had the ability to increase the levels of utrophin protein in dystrophin deficient muscle when compared to vehicle only dosed control animals.

There were two animals injected with up to 50 mg/kg (e.g. 10 mg/kg) of compound administered ip daily for 28 days plus age matched controls. Muscle samples were taken and processed for sectioning (to identify increases in sarcolemmal staining of utrophin) and Western blotting (to identify overall increases in utrophin levels).

FIG. 2 shows an example of TA muscle sections stained with antibody specific for mouse utrophin. Comparison to the mdx muscle only injected with vehicle shows an increase in the amount of sarcolemmal bound utrophin.

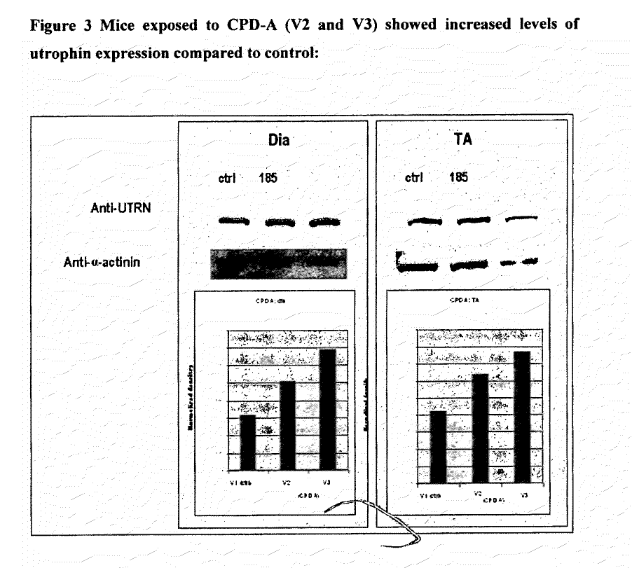

Muscles from the above treated mice were also excised and processed for Western blotting and stained with specific antibodies (see FIG. 3). Again using muscle dosed with CPD-A shows a significant increase in the overall levels of utrophin present in both the TA leg muscle and the diaphragm. Both mice exposed to CPD-A (V2 and V3) showed increased levels of utrophin expression compared to control.

Positive upregulation data from the first 28 day study were then repeated in a further two mouse 28 day study. A total of three different compounds have shown in duplicate the ability to increase the level of utrophin expression in the mdx mouse when delivered daily by ip for 28 days. This data demonstrates the ability of the compound when delivered ip causes a significant increase in the levels of utrophin found in the mdx muscle and therefore gives us the confidence that this approach will ameliorate the disease as all the published data to date demonstrates that any increase of utrophin levels over three fold has significant functional effects on dystrophin deficient muscle.

### H2K/mdx/Utro A Reporter Cell Line Maintenance

The H2K/mdx/Utro A reporter cell line was passaged twice a week until ≦30% confluent. The cells were grown at 33° C. in the presence of 10% CO₂

To remove the myoblasts for platting, they were incubated with Trypsin/EDTA until the monolayer started to detach.

### Growth Medium

DMEM Gibco 41966
20% FCS
1% Pen/Strep
1% glutamine
10 mls Chick embryo extract
Interferon(1276 905 Roche) Add fresh 10 μl/50 mls medium

### Luciferase Assay for 96 Well Plates

The H2K/mdx/Utro A reporter cell line cells were plated out into 96 well plates (Falcon 353296, white opaque) at a density of approximately 5000 cells/well in 190 μl normal growth medium. The plates were then incubated at 33° C. in the presence of 10% CO₂ for 24 hrs.

Compounds were dosed by adding 10 μl of diluted compound to each well giving a final concentration of 10 μM. The plates were then incubated for a further 48 hrs

Cells were then lysed in situ following the manufacture's protocols (Promega Steady-Glo Luciferase Assay System (E2520). Then counted for 10 seconds using a plate luminometer (Victor1420).

### Compound Storage

Compounds for screening were stored at −20° C. as 10 mM stocks in 100% DMSO until required.

Injection of mdx Mice with Compounds

Mdx from a breeding colony were selected for testing. Mice were injected daily with either vehicle or 10 mg/kg of compound using the intreperitoneal route (ip). Mice were weighed and compounds diluted in 5% DMSO, 0.1% tween in PBS.

Mice were sacrificed by cervical dislocation at desired time points, and muscles excised for analysis

### Muscle Analysis

### Immunohistochemistry

Tissues for sectioning were dissected, immersed in OCT (Bright Cryo-M-Bed) and frozen on liquid nitrogen cooled isopentane. Unfixed 8 μM cryosections were cut on a Bright Cryostat, and stored at −80° C.

In readiness for staining, sections were blocked in 5% foetal calf serum in PBS for 30 mins. The primary antibodies were diluted in blocking reagent and incubated on sections for 1.5 hrs in a humid chamber then washed three times for 5 mins in PBS. Secondary antibodies also diluted in blocking reagent, were incubated for 1 hr in the dark in a humid chamber. Finally sections were washed three times 5 mins in PBS and coverslip Mounted with hydromount. Slides were analysed using a Leica fluorescent microscope.

### Results

Biological activity as assessed using the luciferase reporter assay in murine H2K cells, and is classified as follows:

+ Up to 200% relative to control
++ Between 201% and 300% relative to control
+++ Between 301% and 400% relative to control
++++ Above 401% relative to control

TABLE 1
Compounds made by methods described herein

| Example | | |
|---|---|---|
| number | Chemical Name | Activity |
| 1 | N-(2-(4-chlorophenyl)-6-methyl-2H-benzo[d][1,2,3]triazol-5- | +++ |
| | yl)nicotinamide | |
| 2 | N-(2-(4-chlorophenyl)-6-methyl-2H-benzo[d][1,2,3]triazol-5- | ++ |
| | yl)isonicotinamide | |
| 3 | N-(2-(4-chlorophenyl)-6-methyl-2H-benzo[d][1,2,3]triazol-5- | + |
| | yl)benzamide | |
| 4 | N-(2-(4-chlorophenyl)-6-methyl-2H-benzo[d][1,2,3]triazol-5-yl)- | ++ |
| | 4-methoxybenzamide | |
| 5 | N-(2-(4-chlorophenyl)-6-methyl-2H-benzo[d][1,2,3]triazol-5-yl)- | + |
| | 2-methoxybenzamide | |
| 6 | N-(2-(4-chlorophenyl)-6-methyl-2H-benzo[d][1,2,3]triazol-5- | +++ |
| | yl)thiophene-2-carboxamide | |
| 7 | N-(2-(4-chlorophenyl)-6-methyl-2H-benzo[d][1,2,3]triazol-5- | + |
| | yl)propionamide | |
| 8 | N-(2-(4-chlorophenyl)-6-methyl-2H-benzo[d][1,2,3]triazol-5- | ++ |
| | yl)butyramide | |
| 9 | N-(2-(4-chlorophenyl)-6-methyl-2H-benzo[d][1,2,3]triazol-5- | ++ |
| | yl)pentanamide | |
| 10 | N-(2-(4-chlorophenyl)-6-methyl-2H-benzo[d][1,2,3]triazol-5- | ++ |
| | yl)isobutyramide | |
| 11 | N-(2-(4-chlorophenyl)-6-methyl-2H-benzo[d][1,2,3]triazol-5- | ++ |
| | yl)furan-2-carboxamide | |
| 12 | N-(2-(4-(diethylamino)phenyl)-6-methyl-2H- | +++ |
| | benzo[d][1,2,3]triazol-5-yl)nicotinamide | |
| 13 | N-(2-(4-(diethylamino)phenyl)-6-methyl-2H- | +++ |
| | benzo[d][1,2,3]triazol-5-yl)isonicotinamide | |
| 14 | N-(2-(4-(diethylamino)phenyl)-6-methyl-2H- | + |
| | benzo[d][1,2,3]triazol-5-yl)propionamide | |
| 15 | N-(2-(4-(diethylamino)phenyl)-6-methyl-2H- | + |
| | benzo[d][1,2,3]triazol-5-yl)butyramide | |
| 16 | N-(2-(4-(diethylamino)phenyl)-6-methyl-2H- | + |
| | benzo[d][1,2,3]triazol-5-yl)pentanamide | |
| 17 | N-(2-(4-(diethylamino)phenyl)-6-methyl-2H- | + |
| | benzo[d][1,2,3]triazol-5-yl)isobutyramide | |
| 18 | N-(2-(4-(diethylamino)phenyl)-6-methyl-2H- | + |
| | benzo[d][1,2,3]triazol-5-yl)furan-2-carboxamide | |
| 19 | 2-(4-(diethylamino)phenyl)-2H-benzo[d][1,2,3]triazol-5-amine | +++ |
| 20 | N-(2-(4-(diethylamino)phenyl)-2H-benzo[d][1,2,3]triazol-5- | ++ |
| | yl)nicotinamide | |
| 21 | N-(2-(4-(diethylamino)phenyl)-2H-benzo[d][1,2,3]triazol-5- | + |
| | yl)isonicotinamide | |
| 22 | N-(2-(4-(diethylamino)phenyl)-2H-benzo[d][1,2,3]triazol-5- | ++ |
| | yl)acetamide | |
| 23 | N-(2-(4-(diethylamino)phenyl)-2H-benzo[d][1,2,3]triazol-5- | + |
| | yl)propionamide | |
| 24 | N-(2-(4-(diethylamino)phenyl)-2H-benzo[d][1,2,3]triazol-5- | + |
| | yl)butyramide | |
| 25 | N-(2-(4-(diethylamino)phenyl)-2H-benzo[d][1,2,3]triazol-5- | + |
| | yl)pentanamide | |
| 26 | N-(2-(4-(diethylamino)phenyl)-2H-benzo[d][1,2,3]triazol-5- | + |
| | yl)isobutyramide | |
| 27 | N-(2-(4-(diethylamino)phenyl)-2H-benzo[d][1,2,3]triazol-5- | + |
| | yl)furan-2-carboxamide | |
| 28 | N-(2-(4-(diethylamino)phenyl)-2H-benzo[d][1,2,3]triazol-5- | + |
| | yl)thiophene-2-carboxamide | |
| 29 | N-(2-(4-(diethylamino)phenyl)-2H-benzo[d][1,2,3]triazol-5- | + |
| | yl)benzamide | |
| 30 | N-(2-(4-(diethylamino)phenyl)-2H-benzo[d][1,2,3]triazol-5-yl)-4- | + |
| | methoxybenzamide | |
| 31 | N-(2-(4-(diethylamino)phenyl)-2H-benzo[d][1,2,3]triazol-5-yl)-2- | + |
| | methoxybenzamide | |
| 32 | 4-chloro-N-(2-(4-(diethylamino)phenyl)-2H- | + |
| | benzo[d][1,2,3]triazol-5-yl)benzamide | |
| 33 | N-(2-(4-(diethylamino)phenyl)-2H-benzo[d][1,2,3]triazol-5-yl)-4- | + |
| | (dimethylamino)benzamide | |
| 34 | 6-methyl-2-(4-morpholinophenyl)-2H-benzo[d][1,2,3]triazol-5- | ++ |
| | amine | |
| 35 | N-(2-(4-chlorophenyl)-2H-benzo[d][1,2,3]triazol-5- | ++++ |
| | yl)propionamide | |
| 36 | N-(2-(4-chlorophenyl)-2H-benzo[d][1,2,3]triazol-5-yl)butyramide | +++ |
| 37 | N-(2-(4-chlorophenyl)-2H-benzo[d][1,2,3]triazol-5- | ++++ |
| | yl)isobutyramide | |
| 38 | 2-(4-chlorophenyl)-2H-benzo[d][1,2,3]triazol-5-amine | + |
| 39 | N-(2-(4-chlorophenyl)-2H-benzo[d][1,2,3]triazol-5-yl)acetamide | ++++ |
| 40 | 2-(4-(piperidin-1-yl)phenyl)-2H-benzo[d][1,2,3]triazol-5-amine | ++ |
| 41 | 2-(4-(dimethylamino)phenyl)-2H-benzo[d][1,2,3]triazol-5-amine | +++ |
| 42 | 2-(4-(4-methylpiperazin-1-yl)phenyl)-2H-benzo[d][1,2,3]triazol-5- | + |
| | amine | |
| 43 | 2-(4-chlorophenyl)-6-methyl-2H-benzo[d][1,2,3]triazol-5-amine | ++++ |
| 44 | 2-(4-chlorophenyl)-6-(methylsulfonyl)-2H-indazole | ++ |
| 45 | 2-(4-chlorophenyl)-6-nitro-2H-indazole | + |
| 46 | N-(2-(4-chlorophenyl)-2H-indazol-6-yl)isobutyramide | ++++ |
| 47 | 2-(4-chlorophenyl)-6-(methylsulfonyl)-2H-benzo[d][1,2,3]triazole | + |
| | 1-oxide | |
| 48 | 2-(4-chlorophenyl)-2H-indazole | ++++ |
| 49 | 2-(4-chlorophenyl)-5-(methylsulfonyl)-2H-benzo[d][1,2,3]triazole | + |
| 50 | 2-(3,4-dichlorophenyl)-5-(methylsulfonyl)-2H- | ++ |
| | benzo[d][1,2,3]triazole | |
| 51 | 2-(3′,4′-dichlorophenyl)-5-(ethylsulfonyl)-benzotriazole | +++ |
| 52 | 2-(4′-chlorophenyl)-5-(ethylsulfonyl)-benzotriazole | ++++ |
| 53 | N-(2-(3,4-Dichlorophenyl)-2H-benzo[d][1,2,3]triazol-5- | ++++ |
| | yl)isobutyramide | |
| 54 | 6-(Methylsulfonyl)-2-(naphthalen-2-yl)-2H- | ++ |
| | benzo[d][1,2,3]triazole 1-oxide | |
| 55 | 5-(Methylsulfonyl)-2-(naphthalen-2-yl)-2H- | ++++ |
| | benzo[d][1,2,3]triazole | |
| 56 | 2-(4′-Chlorophenyl)-6-(isopropylsulfonyl)-2H-indazole | ++++ |

TABLE 2
Compounds made by analogues methods to those described herein, or by
literature methods known or adapted by the persons skilled in the art.

| Example | | |
|---|---|---|
| number | Chemical Name | Activity |
| 57 | 5-nitro-2-phenyl-2H-benzo[d][1,2,3]triazole | ++ |
| 58 | 2-p-tolyl-2H-benzo[d][1,2,3]triazol-5-amine | + |
| 59 | 2-(4-nitrophenyl)-2H-benzo[d][1,2,3]triazol-5-amine | ++ |
| 60 | 2-(4-methoxyphenyl)-2H-benzo[d][1,2,3]triazol-5-amine | + |
| 61 | 2-(3-chlorophenyl)-2H-benzo[d][1,2,3]triazol-5-amine | + |
| 62 | 2-phenyl-2H-benzo[d][1,2,3]triazol-5-amine | ++ |
| 63 | 2-(3,4-dimethylphenyl)-2H-benzo[d][1,2,3]triazol-5-amine | + |
| 64 | 2-(4-ethoxyphenyl)-6-methyl-2H-benzo[d][1,2,3]triazol-5-amine | ++ |
| 65 | 6-methyl-2-p-tolyl-2H-benzo[d][1,2,3]triazol-5-amine | ++ |
| 66 | N-(2-(4-methoxyphenyl)-6-methyl-2H-benzo[d][1,2,3]triazol-5- | + |
| | yl)acetamide | |
| 67 | N-(6-methyl-2-phenyl-2H-benzo[d][1,2,3]triazol-5-yl)acetamide | ++ |
| 68 | 2-(4-ethylphenyl)-2H-benzo[d][1,2,3]triazol-5-amine | ++ |
| 69 | N-(2-(4-fluorophenyl)-2H-benzo[d][1,2,3]triazol-5-yl)acetamide | ++ |
| 57 | N-(2-(4-Chlorophenyl)-6-methyl-2H-benzo[d][1,2,3]triazol-5- | +++ |
| | yl)acetamide | |
| 58 | 2-(4-Fluorophenyl)-2H-benzo[d][1,2,3]triazol-5-amine | +++ |
| 59 | 2-(4-(Diethylamino)phenyl)-6-methyl-2H-benzo[d][1,2,3]triazol-5- | ++++ |
| | amine | |
| 60 | 2-(5-Amino-2H-benzo[d][1,2,3]triazol-2-yl)phenol | ++++ |
| 61 | 6-Methyl-2-p-tolyl-2H-benzo[d][1,2,3]triazol-5-amine | ++ |
| 62 | 6-Methyl-2-phenyl-2H-benzo[d][1,2,3]triazol-5-amine | ++ |

### EXPERIMENTAL

HPLC-UV-MS was performed on a Gilson 321 HPLC with detection performed by a Gilson 170 DAD and a Finnigan AQA mass spectrometer operating in electrospray ionisation mode. The HPLC column used is a Phenomenex Gemini C18 150×4.6 mm. Preparative HPLC was performed on a Gilson 321 with detection performed by a Gilson 170 DAD. Fractions were collected using a Gilson 215 fraction collector. The preparative HPLC column used is a Phenomenex Gemini C18 150×10 mm and the mobile phase is acetonitrile/water.

¹H NMR spectra were recorded on a Bruker instrument operating at 300 MHz. NMR spectra were obtained as CDCl₃ solutions (reported in ppm), using chloroform as the reference standard (7.25 ppm) or DMSO-D₆ (2.50 ppm). When peak multiplicities are reported, the following abbreviations are used s (singlet), d (doublet), t (triplet), m (multiplet), br (broadened), dd (doublet of doublets), dt (doublet of triplets), td (triplet of doublets). Coupling constants, when given, are reported in Hertz (Hz).

Column chromatography was performed either by flash chromatography (40-65 μm silica gel) or using an automated purification system (SP1™ Purification System from Biotage®). Reactions in the microwave were done in an Initiator 8™ (Biotage).

The abbreviations used are DMSO (dimethylsulfoxide), HCl (hydrochloric acid), MgSO₄ (magnesium sulfate), NaOH (sodium hydroxide), Na₂CO₃ (sodium carbonate), NaHCO₃ (sodium bicarbonate), THF (tetrahydrofuran).

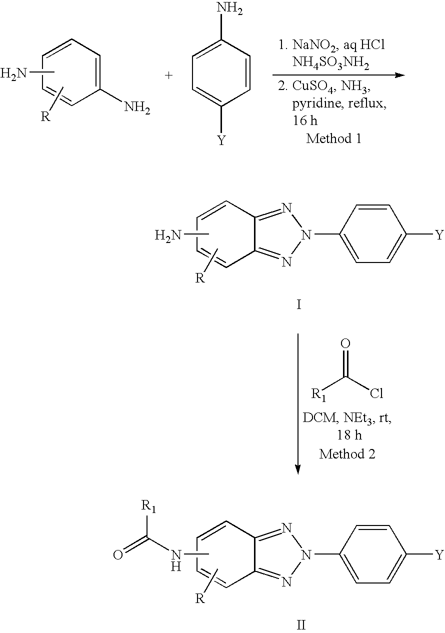

### Method 1

### Compounds I

### 2-(4-(Diethylamino)phenyl)-2H-benzo[d][1,2,3]triazol-5-amine

An aqueous solution (10 mL) of sodium nitrite (764 mg, 11.1 mmol) was added dropwise to a solution of N,N-diethyl-p-phenylenediamine (1.54 mL, 9.3 mmol) in 10% aqueous hydrochloric acid (50 mL) under ice cooling. After 15 min, ammonium sulfamate (1.58 g, 13.8 mmol) was added and the resulting mixture was stirred for 15 min. After adjusting the pH to pH 5 using sodium acetate, 1,3-phenylenediamine (1 g, 9.2 mmol) was added; the mixture was further stirred for 2 h and then basified to pH 9 using 1M sodium hydroxide. Ethyl acetate was added and the organic layer washed twice with brine. The combined organic layers were dried over anhydrous MgSO₄ and evaporated to afford a red solid. A solution of copper sulfate (10 g) in aqueous ammonia (30 mL of 28% ammonia in 30 mL of water) was added to the previously obtained red solid in pyridine (40 mL). The solution was then refluxed for 16 h. After cooling, ethyl acetate was added, and the organic layer washed twice with brine. The combined organic layers were dried over anhydrous MgSO₄ and evaporated down to get a dark red solid, which was triturated with diethyl ether to afford 1.09 g (42%) of the title compound (LCMS RT=7.06 min, MH⁺ 282.1)

¹H NMR (DMSO): 8.02 (2H, d, J 9.3 Hz), 7.68 (1H, d, J 9.1 Hz), 6.96 (1H, dd, J 9.1 2.0 Hz), 6.86 (2H, d, J 9.3 Hz), 6.75 (1H, dd, J 1.9 0.6 Hz), 5.55 (2H, br), 3.46 (4H, q, J 7.1 Hz), 1.19 (6H, t, J 7.1 Hz)

All compounds below were prepared following the same general procedure and purified either by trituration with diethyl ether or by column chromatography on silica gel eluting with a gradient of ethyl acetate/hexanes.

### 6-Methyl-2-(4-morpholinophenyl)-2H-benzo[d][1,2,3]triazol-5-amine

LCMS RT=5.95 min, MH⁺ 311.9; ¹H NMR (DMSO): 8.03 (2H, d, J 9.2 Hz), 7.55 (1H, s), 7.11 (2H, d, J 9.3 Hz), 6.81 (1H, s), 5.32 (2H, s), 3.78-3.75 (4H, m), 3.19-3.16 (4H, m), 2.26 (3H, s)

### 2-(4-Chlorophenyl)-2H-benzo[d][1,2,3]triazol-5-amine

LCMS RT=6.72 min, MH⁺ 245.0; ¹H NMR (DMSO): 8.19 (2H, d, J 9.0 Hz), 7.69 (1H, d, J 9.4 Hz), 7.66 (2H, d, J 9.1 Hz), 6.99 (1H, dd, J 9.1 2.0 Hz), 6.68 (1H, d, J 1.9 Hz), 5.71 (2H, a)

### 2-(4-(Piperidin-1-yl)phenyl)-2H-benzo[d][1,2,3]triazol-5-amine

LCMS RT=7.21 min, MH⁺ 294.2; ¹H NMR (DMSO): 7.99 (2H, d, J 9.2 Hz), 7.65 (1H, d, J 9.2 Hz), 7.08 (2H, d, J 9.2 Hz), 6.92 (1H, dd, J 9.0 1.9 Hz), 6.70-6.69 (1H, m), 5.53 (2H, s), 3.28-3.23 (4H, m), 1.68-1.54 (6H, m)

### 2-(4-(Dimethylamino)phenyl)-2H-benzo[d][1,2,3]triazol-5-amine

LCMS RT=6.13 min, MH⁺ 254.1; ¹H NMR (DMSO): 7.99 (2H, d, J 9.2 Hz), 7.64 (1H, d, J 9.2 Hz), 6.91 (1H, dd, J 9.0 2.0 Hz), 6.86 (2H, d, J 9.2 Hz), 6.70 (1H, d, J 1.5 Hz), 5.50 (2H, s), 2.99 (6H, s)

### 2-(4-(4-Methylpiperazin-1-yl)phenyl)-2H-benzo[d][1,2,3]triazol-5-amine

LCMS RT=4.86 min, MH⁺ 309.1; ¹H NMR (DMSO): 8.01 (2H, d, J 9.2 Hz), 7.65 (1H, d, J 9.2 Hz), 7.10 (2H, d, J 9.2 Hz), 6.93 (1H, dd, J 9.0 1.9 Hz), 6.70-6.69 (1H, m), 5.54 (2H, s), 2.23 (4H, s)

### 2-(4-Chlorophenyl)-6-methyl-2H-benzo[d][1,2,3]triazol-5-amine

LCMS RT=7.13 min, MH⁺ 259.0; ¹H NMR (DMSO): 8.19 (2H, d, J 8.9 Hz), 7.65 (2H, d, J 8.9 Hz), 7.60-7.59 (1H, m), 6.80 (1H, s), 5.48 (2H, s), 2.27 (3H, s)

### Method 2

### Compounds II

### N-(2-(4-Chlorophenyl)-6-methyl-2H-benzo[d][1,2,3]triazol-5-yl)nicotinamide

To a solution of 2-(4-chlorophenyl)-6-methyl-2H-benzo[d][1,2,3]triazol-5-amine (50 mg, 0.19 mmol) and triethylamine (108 μL, 0.77 mmol) in dichloromethane (4 mL) was added 3-nicotinoyl chloride hydrochloride (38 mg, 0.21 mmol). The resulting mixture was stirred at room temperature overnight. Dichloromethane was added and the organic layer was washed twice with aqueous saturated Na₂CO₃. The combined organic layers were dried over anhydrous MgSO₄ and evaporated. The resulting solid was washed with diethyl ether to afford 7 mg (10%) of the title compound (LCMS RT=6.30 min, MH⁺ 364.2)

¹H NMR (DMSO): 10.26 (1H, s), 9.20 (1H, m), 8.82-8.79 (1H, m), 8.39-8.32 (3H, m), 8.13 (1H, s), 7.96 (1H, s), 7.74 (2H, d, J 8.9 Hz), 7.65-7.57 (1H, m)

All compounds below were prepared following the same general procedure.

### N-(2-(4-Chlorophenyl)-6-methyl-2H-benzo[d][1,2,3]triazol-5-yl)isonicotinamide LCMS

RT=6.37 min, MH⁺ 364.0; ¹H NMR (DMSO): 10.33 (1H, s), 8.83 (2H, d, J 6.0 Hz), 8.33 (2H, d, J 8.8 Hz), 8.12 (1H, s), 7.96-7.92 (3H, m), 7.73 (2H, d, J 8.9 Hz)

### N-(2-(4-Chlorophenyl)-6-methyl-2H-benzo[d][1,2,3]triazol-5-yl)benzamide

LCMS RT=7.63 min, MH⁺ 363.1; ¹H NMR (DMSO): 10.05 (1H, s), 8.33 d, J 9.1 Hz), 8.10 (1H, s), 8.04-7.94 (3H, m), 7.73 (2H, d, J 9.1 Hz), 7.64-7.55 (3H, m)

### N-(2-(4-Chlorophenyl)-6-methyl-2H-benzo[d][1,2,3]triazol-5-yl)-4-methoxybenzamide

LCMS RT=7.63 min, MH⁺ 392.7; ¹H NMR (DMSO): 9.88 (1H, s), 8.32 (2H, d, J 9.1 Hz), 8.08 (1H, s), 8.02 (2H, d, J 8.8 Hz), 7.93 (1H, s), 7.73 (2H, d, J 9.0 Hz), 7.09 (2H, d, J 8.8 Hz), 3.86 (3H, s)

### N-(2-(4-Chlorophenyl)-6-methyl-2H-benzo[d][1,2,3]triazol-5-yl)-2-methoxybenzamide

LCMS RT=9.42 min; ¹H NMR (DMSO): 10.23 (1H, s), 8.78 (1H, s), 8.32 (2H, d, J 9.0 Hz), 8.07-8.05 (1H, m), 7.96 (1H, s), 7.72 (2H, d, J 9.0 Hz), 7.66-7.59 (1H, m), 7.33-7.15 (2H, m), 4.08 (3H, s)

### N-(2-(4-Chlorophenyl)-6-methyl-2H-benzo[d][1,2,3]triazol-5-yl)thiophene-2-carboxamide

LCMS RT=7.44 min, MH⁺ 369.0; ¹H NMR (DMSO): 10.07 (1H, s), 8.32 (2H, d, J 9.1 Hz), 8.05-8.03 (2H, m), 7.95 (1H, s), 7.90 (1H, dd, J 5.0 1.0 Hz), 7.72 (2H, d, J 8.9 Hz), 7.28-7.25 (1H, m), 2.45 (3H, s)

### N-(2-(4-Chlorophenyl)-6-methyl-2H-benzo[d][1,2,3]triazol-5-yl)propionamide

LCMS RT=6.86 min, MH⁺ 315.2; ¹H NMR (DMSO): 9.48 (1H, s), 8.47 (2H, d, J 8.9 Hz), 8.32 (1H, s), 8.03 (1H, s), 7.88 (2H, d, J 8.9 Hz), 2.59 (3H, s), 1.30 (3H, t, J 7.1 Hz)

### N-(2-(4-Chlorophenyl)-6-methyl-2H-benzo[d][1,2,3]triazol-5-yl)butyramide

LCMS RT=7.32 min, MH⁺ 329.1; ¹H NMR (DMSO): 9.34 (1H, s), 8.29 (2H, d, J 8.9 Hz), 8.13 (1H, s), 7.86 (1H, s), 7.71 (2H, d, J 8.9 Hz), 2.42 (3H, s), 1.66-1.60 (2H, m), 0.97 (3H, t, J 7.1 Hz)

### N-(2-(4-Chlorophenyl)-6-methyl-2H-benzo[d][1,2,3]triazol-5-yl)pentanamide

LCMS RT=7.82 min, MH⁺ 343.2; ¹H NMR (DMSO): 9.34 (1H, s), 8.30 (2H, d, J 8.9 Hz), 8.13 (1H, s), 7.86 (1H, s), 7.71 (2H, d, J 8.9 Hz), 2.41 (3H, s), 1.66-1.58 (2H, m), 1.42-1.33 (2H, m), 0.94 (3H, t, J 7.1 Hz)

### N-(2-(4-Chlorophenyl)-6-methyl-2H-benzo[d][1,2,3]triazol-5-yl)isobutyramide

LCMS RT=7.23 min, MH⁺ 329.2; ¹H NMR (DMSO): 9.31 (1H, s), 8.30 (2H, d, J 8.9 Hz), 8.09 (1H, s), 7.87 (1H, s), 7.71 (2H, d, J 8.9 Hz), 2.77-2.73 (1H, m), 2.41 (3H, s), 1.17 (6H, d, J 6.8 Hz)

### N-(2-(4-Chlorophenyl)-6-methyl-2H-benzo[d][1,2,3]triazol-5-yl)furan-2-carboxamide

LCMS RT=7.44 min, MH⁺ 353.1; ¹H NMR (DMSO): 9.89 (1H, s), 8.32 (2H, d, J 9.1 Hz), 8.10 (1H, s), 7.98-7.93 (2H, m), 7.73 (2H, d, J 8.9 Hz), 7.36 (1H, dd, J 3.5 0.8 Hz), 6.74 (1H, dd, J 3.5 1.8 Hz), 2.44 (3H, s)

### N-(2-(4-(Diethylamino)phenyl)-6-methyl-2H-benzo[d][1,2,3]triazol-5-yl)nicotinamide

LCMS RT=6.94 min, MH⁺ 401.0; ¹H NMR (DMSO): 10.23 (1H, s), 9.20 (1H, m), 8.81-8.78 (1H, m), 8.39-8.33 (1H, m), 8.08 (2H, d, J 9.1 Hz), 8.01 (1H, s), 7.88-7.86 (1H, m), 7.63-7.56 (1H, m), 6.85 (2H, d, J 9.2 Hz), 3.43-3.40 (4H, m), 1.15 (6H, t, J 6.7 Hz)

### N-(2-(4-(Diethylamino)phenyl)-6-methyl-2H-benzo[d][1,2,3]triazol-5-yl)isonicotinamide

LCMS RT=6.99 min, MH⁺ 400.9; ¹H NMR (DMSO): 10.31 (1H, s), 8.82 (2H, d, J 6.0 Hz), 8.08 (2H, d, J 9.2 Hz), 8.00 (1H, s), 7.93 (2H, d, J 5.9 Hz), 7.89-7.87 (1H, m), 6.85 (2H, d, J 9.2 Hz), 3.42-3.38 (4H, m), 2.43 (3H, s), 1.15 (6H, t, J 7.2 Hz)

### N-(2-(4-(Diethylamino)phenyl)-6-methyl-2H-benzo[d][1,2,3]triazol-5-yl)propionamide

LCMS RT=7.51 min, M⁺ 352.2; ¹H NMR (DMSO): 9.28 (1H, s), 8.06-8.02 (3H, m), 7.78 (1H, s), 6.83 (2H, d, J 9.2 Hz), 3.42-3.37 (6H, m), 2.38 (3H, s), 1.16-1.10 (9H, m)

### N-(2-(4-(Diethylamino)phenyl)-6-methyl-2H-benzo[d][1,2,3]triazol-5-yl)butyramide

LCMS RT=7.97 min, MH⁺ 366.1; ¹H NMR (DMSO): 9.32 (1H, s), 8.04 (2H, d, J 8.9 Hz), 8.00 (1H, s), 7.78 (1H, s), 6.84 (2H, d, J 8.9 Hz, 1.70-1.62 (2H, m), 1.17-1.07 (6H, m), 0.97 (3H, t, J 7.1 Hz)

### N-(2-(4-(Diethylamino)phenyl)-6-methyl-2H-benzo[d][1,2,3]triazol-5-yl)pentanamide

LCMS RT=8.53 min, MH⁺ 379.9; ¹H NMR (DMSO): 9.32 (1H, s), 8.04 (2H, d, J 8.9 Hz), 8.00 (1H, s), 7.78 (1H, s), 6.83 (2H, d, J 8.9 Hz), 3.50-3.35 (6H, m), 2.39 (3H, s), 1.65-1.61 (2H, m), 1.41-1.34 (2H, m), 1.17-1.07 (6H, m), 0.94 (3H, t, J 7.1 Hz)

### N-(2-(4-(Diethylamino)phenyl)-6-methyl-2H-benzo[d][1,2,3]triazol-5-yl)isobutyramide

LCMS RT=7.92 min, MH⁺ 366.1; ¹H NMR (DMSO): 9.29 (1H, s), 8.05 (2H, d, J 8.9 Hz), 7.96 (1H, s), 7.78 (1H, s), 6.84 (2H, d, J 8.9 Hz), 3.42-3.37 (4H, m), 2.41 (3H, s), 1.17-1.07 (12H, m)

### N-(2-(4-(Diethylamino)phenyl)-6-methyl-2H-benzo[d][1,2,3]triazol-5-yl)furan-2-carboxamide

LCMS RT=8.18 min, MH⁺ 389.9; ¹H NMR (DMSO): 9.87 (1H, s), 8.07 (2H, d, J 9.1 Hz), 7.98 (1H, s), 7.98-7.96 (1H, m), 7.85 (1H, m), 7.34 (1H, dd, J 3.4 0.7 Hz), 6.87 (2H, d, J 9.2 Hz), 6.73 (1H, dd, J 3.5 1.7 Hz), 3.42-3.37 (4H, m), 2.41 (3H, s), 1.17-1.07 (6H, m)

### N-(2-(4-(Diethylamino)phenyl)-2H-benzo[d][1,2,3]triazol-5-yl)nicotinamide

LCMS RT=7.19 min, MH⁺ 387.1; ¹H NMR (DMSO): 10.69 (1H, s), 9.16 (1H, s), 8.82-8.77 (1H, m), 8.54 (1H, s), 8.34 (1H, d, J 7.8 Hz), 8.08 (2H, d, J 9.3 Hz), 7.98 (1H, d, J 9.3 Hz), 7.71 (1H, dd, J 9.1 1.7 Hz), 7.58-7.53 (1H, m), 6.85 (2H, d, J 9.2 Hz), 3.44 (4H, q, J 6.8 Hz), 1.18 (6H, t, J 6.8 Hz)

### N-(2-(4-(Diethylamino)phenyl)-2H-benzo[d][1,2,3]triazol-5-yl)isonicotinamide

LCMS RT=7.23 min, MH⁺ 387.1; ¹H NMR (DMSO): 10.74 (1H, s), 8.82 (2H, d, J 5.6 Hz), 8.58-8.53 (1H, m), 8.08 (2H, d, J 9.4 Hz), 7.98 (1H, dd, J 9.1 0.6 Hz), 7.91 (2H, d, J 6.1 Hz), 7.71 (1H, dd, J 9.1 1.8 Hz), 6.86 (2H, d, J 9.1 Hz), 3.43 (4H, q, J 7.1 Hz), 1.15 (6H, t, J 7.1 Hz)

### N-(2-(4-(Diethylamino)phenyl)-2H-benzo[d][1,2,3]triazol-5-yl)acetamide

LCMS RT=6.89 min, MH⁺ 324.2; ¹H NMR (DMSO): 10.20 (1H, s), 8.38 (1H, d, J 1.8 Hz), 8.04 (2H, d, J 9.2 Hz), 7.89 (1H, dd, J 9.1 0.6 Hz), 7.42 (1H, dd, J 9.2 1.8 Hz), 6.84 (2H, d, J 9.2 Hz), 3.43 (4H, q, J 6.9 Hz), 2.11 (3H, s), 1.14 (6H, t, J 6.9 Hz)

### N-(2-(4-(Diethylamino)phenyl)-2H-benzo[d][1,2,3]triazol-5-yl)propionamide

LCMS RT=7.50 min, MH⁺ 338.2; ¹H NMR (DMSO): 10.12 (1H, s), 8.41 (1H, d, J 1.0 Hz), 8.04 (2H, d, J 9.2 Hz), 7.89 (1H, d, J 9.2 Hz), 7.44 (1H, dd, J 9.1 1.8 Hz), 6.84 (2H, d, J 9.4 Hz), 3.42 (4H, q, J 6.9 Hz), 2.39 (2H, q, J 7.4 Hz), 1.17-1.09 (9H, m)

### N-(2-(4-(Diethylamino)phenyl)-2H-benzo[d][1,2,3]triazol-5-yl)butyramide

LCMS RT=8.00 min, MH⁺ 352.1; ¹H NMR (DMSO): 10.13 (1H, s), 8.42-8.40 (1H, m), 8.04 (2H, d, J 9.2 Hz), 7.89 (1H, d, J 9.2 Hz), 7.44 (1H, dd, J 9.1 1.7 Hz), 6.84 (2H, d, J 9.4 Hz), 3.42 (4H, q, J 6.9 Hz), 2.36 (2H, q, J 7.4 Hz), 1.72-1.60 (2H, m), 1.14 (6H, t, J 7.0 Hz), 0.95 (3H, t, J 7.4 Hz)

### N-(2-(4-(Diethylamino)phenyl)-2H-benzo[d][1,2,3]triazol-5-yl)pentanamide

LCMS RT=8.60 min, MH⁺ 365.9; ¹H NMR (DMSO): 10.13 (1H, s), 8.42-8.40 (1H, m), 8.04 (2H, d, J 9.2 Hz), 7.89 (1H, d, J 9.2 Hz), 7.44 (1H, dd, J 9.1 1.7 Hz), 6.84 (2H, d, J 9.4 Hz), 3.42 (4H, q, J 6.9 Hz), 2.38 (2H, q, J 7.4 Hz), 1.67-1.57 (2H, m), 1.42-130 (2H, m), 1.14 (6H, t, J 7.0 Hz), 0.92 (3H, t, J 7.4 Hz)

### N-(2-(4-(Diethylamino)phenyl)-2H-benzo[d][1,2,3]triazol-5-yl)isobutyramide

LCMS RT=7.95 min, MH⁺ 352.2; ¹H NMR (DMSO): 10.09 (1H, s), 8.42-8.35 (1H, m), 8.04 (2H, d, J 9.2 Hz), 7.89 (1H, d, J 9.2 Hz), 7.46 (1H, dd, J 9.1 1.8 Hz), 6.84 (2H, d, J 9.4 Hz), 3.42 (4H, q, J 6.9 Hz), 2.70-2.61 (1H, m), 1.18-1.12 (12H, m)

### N-(2-(4-(Diethylamino)phenyl)-2H-benzo[d][1,2,3]triazol-5-yl)furan-2-carboxamide

LCMS RT=7.98 min, MH⁺ 376.3; ¹H NMR (DMSO): 10.43 (1H, s), 8.48-8.47 (1H, m), 8.07 (2H, d, J 9.2 Hz), 7.99-7.98 (1H, m), 7.94 (1H, d, J 9.2 Hz), 7.74 (1H, dd, J 9.3 1.9 Hz), 7.40 (1H, d, J 3.5 Hz), 6.85 (2H, d, J 9.3 Hz), 6.75-6.72 (1H, m), 3.43 (4H, q, J 7.1 Hz), 1.15 (6H, t, J 7.0 Hz)

### N-(2-(4-(Diethylamino)phenyl)-2H-benzo[d][1,2,3]triazol-5-yl)thiophene-2-carboxamide

LCMS RT=8.47 min, MH⁺ 391.9; ¹H NMR (DMSO): 10.45 (1H, s), 8.46-8.45 (1H, m), 8.09-8.06 (3H, m), 7.96 (1H, d, J 9.3 Hz), 7.91 (1H, dd, J 5.0 1.0 Hz), 7.70 (1H, dd, J 9.2 1.8 Hz), 7.28-7.25 (1H, m), 6.84 (2H, d, J 9.4 Hz), 3.43 (4H, q, J 7.0 Hz), 1.15 (6H, t, J 7.0 Hz)

### N-(2-(4-(Diethylamino)phenyl)-2H-benzo[d][1,2,3]triazol-5-yl)benzamide

LCMS RT=8.62 min, MH⁺ 385.9; ¹H NMR (DMSO): 10.50 (1H, s), 8.54-8.53 (1H, m), 8.08 (2H, d, J 9.2 Hz), 8.02-7.99 (2H, m), 7.96 (1H, dd, J 9.1 0.6 Hz), 7.74 (1H, dd, J 9.2 1.8 Hz), 7.67-7.54 (3H, m), 6.85 (2H, d, J 9.3 Hz), 3.44 (4H, q, J 7.0 Hz), 1.15 (611, t, J 7.0 Hz)

### N-(2-(4-(Diethylamino)phenyl)-2H-benzo[d][1,2,3]triazol-5-yl)-4-methoxybenzamide

LCMS RT=8.58 min, MH⁺ 416.2; ¹H NMR (DMSO): 10.33 (1H, s), 8.52-8.51 (1H, m), 8.06 (2H, d, J 9.2 Hz), 8.01 (2H, d, J 8.8 Hz), 7.94 (1H, d, J 9.2 Hz), 7.73 (1H, dd, J 9.2 1.8 Hz), 7.09 (2H, d, J 8.8 Hz), 6.85 (2H, d, J 9.3 Hz), 3.86 (3H, s), 3.43 (4H, q, J 7.0 Hz), 1.15 (6H, t, J 7.0 Hz)

### N-(2-(4-(Diethylamino)phenyl)-2H-benzo[d][1,2,3]triazol-5-yl)-2-methoxybenzamide

LCMS RT=9.56 min, MH⁺ 415.9; ¹H NMR (DMSO): 10.40 (1H, s), 8.56-8.55 (1H, m), 8.07 (2H, d, J 9.2 Hz), 7.93 (1H, d, J 9.2 Hz), 7.67 (1H, dd, J 7.5 1.6 Hz), 7.61 (1H, dd, J 9.1 1.7 Hz), 7.56-7.51 (1H, m), 7.21 (1H, d, J 8.6 Hz), 7.10 (1H, t, J 7.5 Hz), 6.85 (2H, d, J 9.3 Hz), 3.93 (3H, s), 3.43 (4H, q, J 7.0 Hz), 1.15 (6H, t, J 7.0 Hz)

### 4-Chloro-N-(2-(4-(diethylamino)phenyl)-2H-benzo[d][1,2,3]triazol-5-yl)benzamide

LCMS RT=9.71 min, MH⁺ 420.0; ¹H NMR (DMSO): 10.56 (1H, s), 8.53-8.52 (1H, m), 8.08 (2H, d, J 9.2 Hz), 8.04 (2H, d, J 8.7 Hz), 7.96 (1H, d, J 9.1 Hz), 7.72 (1H, dd, J 9.2 1.8 Hz), 7.65 (2H, d, J 8.6 Hz), 6.85 (2H, d, J 9.2 Hz), 3.44 (4H, q, J 7.0 Hz), 1.15 (6H, t, J 7.0 Hz)

### N-(2-(4-(Diethylamino)phenyl)-2H-benzo[d][1,2,3]triazol-5-yl)-4-(dimethylamino)benzamide

LCMS RT=8.84 min, MH⁺ 428.9; ¹H NMR (DMSO): 10.02 (1H, s), 8.43-8.42 (1H, m), 7.99 (2H, d, J 9.2 Hz), 7.85-7.82 (3H, m), 7.66 (1H, dd, J 9.1 1.7 Hz), 6.77 (2H, d, J 9.4 Hz), 6.71 (2H, d, J 9.1 Hz), 3.35 (4H, q, J 7.0 Hz), 2.94 (6H, s), 1.07 (6H, t, J 7.0 Hz)

### N-(2-(4-Chlorophenyl)-2H-benzo[d][1,2,3]triazol-5-yl)propionamide

LCMS RT=7.16 min, MH⁺ 301.0; ¹H NMR (DMSO): 10.22 (1H, s), 8.50-8.48 (1H, m), 8.30 (2H, d, J 9.0 Hz), 7.97 (1H, d, J 9.3 Hz), 7.71 (2H, d, J 9.0 Hz), 7.50 (1H, dd, J 9.3 1.7 Hz), 1.13 (3H, t, J 7.1 Hz)

### N-(2-(4-Chlorophenyl)-2H-benzo[d][1,2,3]triazol-5-yl)butyramide

LCMS RT=7.64 min, MH⁺ 314.8; ¹H NMR (DMSO): 10.22 (1H, s), 8.49-8.48 (1H, m), 8.29 (2H, d, J 9.0 Hz), 7.97 (1H, d, J 9.3 Hz), 7.71 (2H, d, J 9.0 Hz), 7.51 (1H, dd, J 9.3 1.7 Hz), 2.38 (2H, t, J 7.0 Hz), 1.72-1.59 (2H, m), 0.94 (3H, t, J 7.4 Hz)

### N-(2-(4-Chlorophenyl)-2H-benzo[d][1,2,3]triazol-5-yl)isobutyramide

LCMS RT=7.59 min, MH⁺ 314.9; ¹H NMR (DMSO): 10.18 (1H, s), 8.50-8.49 (1H, m), 8.30 (2H, d, J 9.0 Hz), 7.97 (1H, d, J 9.3 Hz), 7.71 (2H, d, J 9.0 Hz), 7.53 (1H, dd, J 9.3 1.7 Hz), 2.67 (1H, m), 1.15 (6H, d, J 6.8 Hz)

### N-(2-(4-Chlorophenyl)-2H-benzo[d][1,2,3]triazol-5-yl)acetamide

LCMS RT=6.52 min, MH⁺ 287.0; ¹H NMR (DMSO): 10.30 (1H, s), 8.47-8.45 (1H, m), 8.29 (2H, d, J 9.0 Hz), 7.98 (1H, d, J 9.3 Hz), 7.71 (2H, d, J 9.0 Hz), 7.49 (1H, dd, J 9.3 1.7 Hz), 2.13 (3H, s)

### N-(2-(3,4-Dichlorophenyl)-2H-benzo[d][1,2,3]triazol-5-yl)isobutyramide

LCMS RT=8.29 min, MH⁺ 349.1; ¹H NMR (DMSO): 10.20 (1H, s), 8.50 (1H, dd, J 1.8 0.7 Hz), 8.48 (1H, d, J 2.5 Hz), 8.27 (1H, dd, J 8.8 2.5 Hz), 7.98 (1H, dd, J 9.2 0.7 Hz), 7.92 (1H, d, J 8.8 Hz), 7.55 (1H, dd, J 9.3 1.8 Hz), 2.71-2.62 (1H, m), 1.15 (6H, d, J 6.8 Hz)

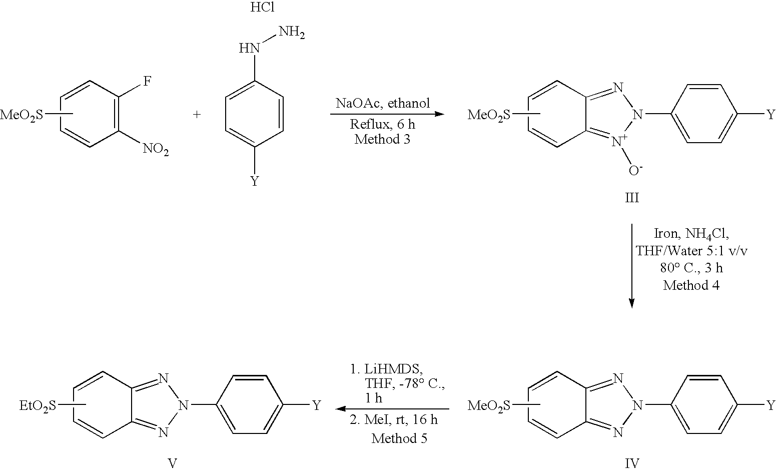

### Method 3

### Compounds III

### 2-(4-Chlorophenyl)-6-(methylsulfonyl)-2H-benzo[d][1,2,3]triazole 1-oxide

(4-Chlorophenyl)hydrazine hydrochloride (1.64 g, 9.17 mmol), 1-fluoro-4-(methylsulfonyl)-2-nitrobenzene (1.00 g, 4.56 mmol) and sodium acetate trihydrate (1.87 g, 13.7 mmol) were suspended in ethanol (15 mL) and heated to reflux for 6 h. The mixture was then cooled to room temperature and the product removed by filtration. The residue was washed with methanol, water and then methanol again to afford 1.13 g (77%) of the title compound (LCMS RT=5.92 min, (MH⁺+MeCN) 364.9)

¹H NMR (DMSO): 8.39-8.38 (1H, m), 8.21-8.14 (3H, m), 7.98 (1H, dd, J 9.2 1.7 Hz), 7.80 (2H, d, J 9.0 Hz), 3.38 (3H, s)

### 6-(Methylsulfonyl)-2-(naphthalen-2-yl)-2H-benzo[d][1,2,3]triazole 1-oxide

LCMS RT=6.12 min; ¹H NMR (DMSO): 8.84 (1H, d, J 1.8 Hz), 8.42-8.41 (1H, m), 8.27-8.10 (5H, m), 8.01 (1H, dd, J 9.2 1.7 Hz), 7.76-7.68 (2H, m), 3.39 (3H, s)

### Method 4

### Compounds IV

### 2-(4-Chlorophenyl)-5-(methylsulfonyl)-2H-benzo[d][1,2,3]triazole

To a suspension of 2-(4-chlorophenyl)-6-(methylsulfonyl)-2H-benzo[d][1,2,3]triazole 1-oxide (157 mg, 0.49 mmol) and ammonium chloride (52 mg, 0.97 mmol) in tetrahydrofuran/water 5:1 v/v (6 mL) at 80° C. was added iron powder (136 mg, 2.43 mmol). The resulting mixture was stirred for 3 h at 80° C. After cooling, the solution was passed through a pad of Celite® and washed with tetrahydrofuran. The filtrate was then concentrated in vacuo, suspended in water and extracted three times with ethyl acetate. The combined organic layers were dried over anhydrous MgSO₄ and evaporated. The resulting solid was purified by column chromatography eluting with ethyl acetate/hexanes 25:75 v/v to afford 29.7 mg (20%) of the title compound (LCMS RT=6.59 min)

¹H NMR (CDCl₃): 8.60-8.58 (1H, m), 8.28 (2H, d, J 9.0 Hz), 8.04 (1H, dd, J 9.0 0.9 Hz), 7.82 (1H, dd, J 9.0 1.6 Hz), 7.49 (2H, d, J 9.0 Hz), 3.06 (3H, s)

The compound below was prepared following the same general procedure.

### 2-(3,4-Dichlorophenyl)-5-(methylsulfonyl)-2H-benzo[d][1,2,3]triazole

LCMS RT=7.35 min, MH⁺ 342.1; ¹H NMR (DMSO): 8.70-8.69 (1H, m), 8.57 (1H, d, J 2.5 Hz), 8.37-8.33 (2H, m), 8.04-7.97 (2H, m), 3.37 (3H, s)

### 5-(Methylsulfonyl)-2-(naphthalen-2-yl)-2H-benzo[d][1,2,3]triazole

LCMS RT=6.92 min; ¹H NMR (DMSO): 9.01 (1H, d, J 2.1 Hz), 8.73-8.72 (1H, m), 8.52 (1H, dd, J 8.9 2.2 Hz), 8.38 (1H, dd, J 9.0 0.8 Hz), 8.27 (2H, d, J 8.6 Hz), 8.13-8.08 (1H, m), 8.02 (1H, dd, J 9.0 1.7 Hz), 7.71-7.67 (2H, m), 3.38 (3H, s)

### Method 5

### Compounds V

### 2-(3,4-Dichlorophenyl)-5-(ethylsulfonyl)-2H-benzo[d][1,2,3]triazole

To a dry Schlenk flask under nitrogen was added 2-(3,4-dichlorophenyl)-5-(methylsulfonyl)-2H-benzo[d][1,2,3]triazole (93.5 mg, 0.27 mmol) and dry tetrahydrofuran (5 mL). The solution was then cooled down to −78° C., and lithium bis(trimethylsilyl)amide (0.30 mL, 0.30 mmol) was added. The reaction was left stirring at −78° C. for 1 h, and then methyl iodide (35 μL, 0.55 mmol) was added. The solution was allowed to warm up to room temperature for 16 h. Aqueous saturated ammonium chloride (10 mL) was added to the solution, the organic layer was separated and the aqueous layer was extracted three times with ethyl acetate. The combined organic layers were dried over anhydrous MgSO₄ and evaporated. The resulting solid was purified by column chromatography eluting with ethyl acetate/hexanes 20:80 v/v to afford 52 mg (54%) of the title compound (LCMS RT=7.65 min)

¹H NMR (DMSO): 8.68-8.67 (1H, m), 8.57 (1H, d, J 2.5 Hz), 8.38-8.33 (2H, m), 8.01-7.94 (2H, m), 3.46 (2H, q, J 7.5 Hz), 1.15 (3H, t, J 7.4 Hz)

The compound below was prepared following the same general procedure.

### 2-(4-Chlorophenyl)-5-(ethylsulfonyl)-2H-benzo[d][1,2,3]triazole

LCMS RT=6.89 min; ¹H NMR (DMSO): 8.67-8.66 (1H, m), 8.39 (2H, d, J 9.1 Hz), 8.34 (1H, dd, J 9.0 0.8 Hz), 7.95 (1H, dd, J 9.0 1.6 Hz), 7.79 (2H, d, J 9.0 Hz), 3.45 (2H, q, J 7.3 Hz), 1.15 (3H, t, J 7.4 Hz)

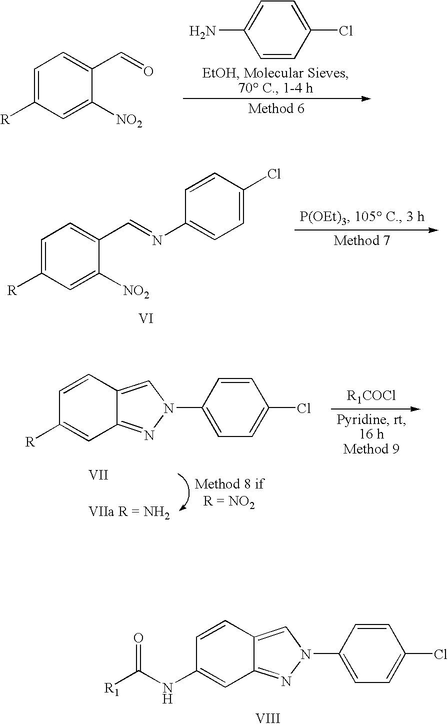

### Method 6

### Compounds VI

### (E)-4-Chloro-N-(4-(methylsulfonyl)-2-nitrobenzylidene)aniline

To 4-(methylsulfonyl)-2-nitrobenzaldehyde (250 mg, 1.09 mmol) in ethanol (5 mL) with molecular sieves at room temperature was added 4-chloroaniline (139 mg, 1.09 mmol). The resulting mixture was stirred at room temperature for 1 h, and then heated at 70° C. for 1 h. After cooling, the mixture was filtered off, and the filtrate concentrated in vacuo to afford the title compound, which was used crude in the next step.

### Method 7

### Compounds VII

### 2-(4-Chlorophenyl)-6-(methylsulfonyl)-2H-indazole

A suspension of (E)-4-Chloro-N-(4-(methylsulfonyl)-2-nitrobenzylidene)aniline (133 mg, 0.39 mmol) in triethyl phosphate (2 mL) was stirred at 105° C. for 3 h. After cooling, a solid was filtered off and washed with hexanes to afford 89 mg (74%) of the title compound (LCMS RT=6.17 min, MH⁺ 307.0)

¹H NMR (DMSO): 9.36 (1H, d, J 0.9 Hz), 8.34 (1H, br), 8.19 (2H, d, J 8.9 Hz), 8.08 (1H, dd, J 8.9 0.8 Hz), 7.73 (2H, d, J 8.9 Hz), 7.58 (1H, dd, J 8.8 1.4 Hz), 3.30 (3H, s)

The compound below was prepared following the same general procedure.

### 2-(4-Chlorophenyl)-6-nitro-2H-indazole

LCMS RT=7.27 min; ¹H NMR (DMSO): 9.40 (1H, s), 8.76-8.74 (1H, m), 8.20 (2H, d, J 9.0 Hz), 8.08 (1H, d, J 9.2 Hz), 7.89 (1H, dd, J 9.2 2.0 Hz), 7.74 (2H, d, J 8.9 Hz)

### 2-(4-Chlorophenyl)-2H-indazole

LCMS RT=7.05 min, MH⁺ 229.0; ¹H NMR (DMSO): 9.14 (1H, d, J 0.9 Hz), 8.14 (2H, d, J 9.0 Hz), 7.77 (1H, dt, J 8.4 1.1 Hz), 7.71 (1H, dd, J 8.8 0.9 Hz), 7.67 (2H, d, J 9.0 Hz), 7.33 (1H, ddd, J 8.9 6.6 1.1 Hz), 7.12 (1H, ddd, J 8.4 6.6 0.8 Hz)

### Method 8

### Compounds VIIa

### 2-(4-Chlorophenyl)-2H-Indazol-6-amine

To 2-(4-chlorophenyl)-6-nitro-2H-indazole (103 mg, 0.37 mmol) in tetrahydrofuran:water 4:1 v/v (5 mL) at room temperature was added ammonium chloride (40 mg, 0.75 mmol). The mixture was heated at 80° C. and iron powder (105 mg, 1.87 mmol) was added. The resulting mixture was stirred at 80° C. for 3 h. After cooling, the solution was filtered through a pad of Celite® and washed with tetrahydrofuran. After evaporation of the solvent, the aqueous layer was extracted twice with ethyl acetate. The combined organic layers were washed with brine, dried over anhydrous MgSO₄ and evaporated to afford 84 mg (92%) of the title compound.

### Method 9

### Compounds VIII

### N-(2-(4-Chlorophenyl)-2H-indazol-6-yl)isobutyramide

To a solution of 2-(4-chlorophenyl)-2H-indazol-6-amine (84 mg, 0.34 mmol) in pyridine (5 mL) at room temperature was added isobutyryl chloride (43 μL, 0.41 mmol). The resulting mixture was stirred at room temperature for 16 h. Ethyl acetate was added and the organic layer was washed twice with saturated aqueous copper sulfate, followed by brine and water. The combined organic layers were dried over anhydrous MgSO₄ and evaporated. The resulting solid was purified by column chromatography eluting with ethyl acetate/hexanes 25:75 v/v to afford 11 mg (10%) of the title compound (LCMS RT=6.38 min, MH⁺ 314.2)

¹H NMR (DMSO): 10.14 (1H, s), 9.24 (1H, d, J 0.8 Hz), 8.42-8.40 (1H, m), 8.31 (2H, d, J 9.0 Hz), 7.89 (1H, dd, J 9.1 0.7 Hz), 7.85 (2H, d, J 8.9 Hz), 7.36 (1H, dd, J 9.0 1.6 Hz), 2.90-2.81 (1H, m), 1.34 (6H, d, J 6.8 Hz)

### Method 10

### Compounds IX

### 2-(4′-Chlorophenyl)-6-(isopropylsulfonyl)-2H-indazole

To a dry Schlenk flask under nitrogen was added 2-(4-chlorophenyl)-6-(methylsulfonyl)-2H-indazole (200 mg, 0.65 mmol) and dry tetrahydrofuran (9 mL). The solution was then cooled down to −78° C., and lithium bis(trimethylsilyl)amide (0.72 mL, 0.72 mmol) was added. The reaction was left stirring at −78° C. for 1 h, and then methyl iodide (81 μL, 1.31 mmol) was added. The solution was allowed to warm up to room temperature for 16 h. Aqueous saturated ammonium chloride (10 mL) was added to the solution, the organic layer was separated and the aqueous layer was extracted three times with ethyl acetate. The combined organic layers were dried over anhydrous MgSO₄ and evaporated. The resulting solid was purified by column chromatography eluting with ethyl acetate/hexanes 1:2 v/v to afford 20 mg (9%) of the title compound (LCMS RT=6.53 min, MH⁺ 335.2)

¹H NMR (DMSO): 9.37 (1H, d, J 0.9 Hz), 8.29-8.27 (1H, m), 8.18 (2H, d, J 9.0 Hz), 8.07 (1H, dd, J 8.8 0.8 Hz), 7.72 (2H, d, J 9.0 Hz), 7.49 (1H, dd, J 8.8 1.6 Hz), 3.58-3.48 (1H, m), 1.20 (6H, d, J 6.8 Hz)

The compounds listed in Table 2, can be prepared by analogues methods to those described above, or by literature methods known or adapted by the persons skilled in the art.

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1. A combination comprising an ancillary agent and a compound of Formula (I) or (II): 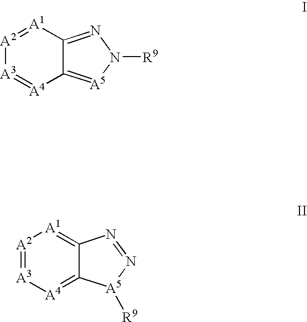
wherein
A¹, A², A³, A⁴ and A⁵, which may be the same or different, each represent N or CR¹;
R₉ represents -L-R³, in which L is a single bond or a linker group and R³ represents hydrogen or a substituent;
when an adjacent pair of A¹-A⁴ each represent CR¹, the adjacent carbon atoms, together with their substituents, may form a ring B; and
when A⁵ represents CR¹ in formula (I), A⁵ and N—R⁹, together with their substituents, may form a ring C;
or a pharmaceutically acceptable salt thereof.

2. The combination according to claim 1, wherein R³ represents alkyl, alkoxy or aryl, each optionally substituted by one to three substituents, which may be the same or different.

3. The combination according to claim 1, wherein
(i) in the compound of formula I or formula II
A⁵ represents N;
L is single bond; and
R³ represents:
thioalkyl optionally substituted by alkyl or optionally substituted aryl,
O-aryl or thioaryl, in which the aryl is optionally substituted,
optionally substituted aryl,
hydroxyl,
NR¹⁰R¹¹,
SO₂R¹²,
NR¹³SO₂R¹⁴,
C(═W)R¹⁶, or
NR¹⁵C(═W)R¹⁷; wherein
R¹⁰, R¹¹, R¹², R¹³, R¹⁴, R¹⁵, R¹⁶ and R¹⁷, which may be the same or different, each represent hydrogen, alkyl optionally substituted by optionally substituted aryl, or optionally substituted aryl;
in addition,
R¹⁰ and R¹¹ together with the nitrogen to which they are attached may form a ring;
R¹² may have the same meaning as NR¹⁰R¹¹;
R¹⁶ and R¹⁷, which may be the same or different, may each represent
alkyl optionally substituted by one or more of halogen, alkoxy optionally substituted aryl or optionally substituted aryl,
optionally substituted aryloxy,
aryl or NR¹⁰R¹¹; or
when R¹⁶ or R¹⁷ represents NR¹⁰R¹¹, one of R¹⁰ and R¹¹ may additionally represent optionally substituted COalkyl or optionally substituted COaryl;
R¹⁶ may additionally represent hydroxyl; and
W is O;
or (ii) in the compound of formula II
A⁵ represents CH;
L is single bond; and
R³ represents:
thioalkyl optionally substituted by alkyl or optionally substituted aryl,
thioaryl, in which the aryl is optionally substituted,
optionally substituted aryl,
hydroxyl,
NO₂,
CN,
NR¹⁰R¹¹,
halogen,
SO₂R¹²,
NR¹³SO₂R¹⁴,
C(═W)R¹⁶,
OC(═W)NR¹⁰R¹¹, or
NR¹⁵C(═W)R¹⁷; wherein
R¹⁰, R¹¹, R¹², R¹³, R¹⁴, R¹⁵, R¹⁶ and R¹⁷, which may be the same or different, each represent hydrogen, alkyl optionally substituted by optionally substituted aryl, or optionally substituted aryl;
in addition,
R¹⁰ and R¹¹ together with the nitrogen to which they are attached may form a ring;
R¹² may have the same meaning as NR¹⁰R¹¹;
R¹⁶ and R¹⁷, which may be the same or different, may each represent
alkyl optionally substituted by one or more of halogen, alkoxy optionally substituted aryl or optionally substituted aryl,
optionally substituted aryloxy,
aryl or NR¹⁰R¹¹; or
when R¹⁶ or R¹⁷ represents NR¹⁰R¹¹, one of R¹⁰ and R¹¹ may additionally represent optionally substituted COalkyl or optionally substituted COaryl; and
R¹⁶ may additionally represent hydroxyl; and
W is O.

4. The combination according to claim 2, in which the substituent(s) on R³ is R², and R¹ and R², which may be the same or different, may each independently represent:
alkyl optionally substituted by one or more halogen, alkoxy or optionally substituted aryl, thioaryl or aryloxy,
alkoxy optionally substituted by optionally by alkyl or optionally substituted aryl,
hydroxyl,
OC(═W)NR¹⁰R¹¹,
aryl,
thioalkyl optionally substituted by alkyl or optionally substituted aryl,
thioaryl, in which the aryl is optionally substituted,
NO₂,
CN,
NR¹⁰R¹¹,
halogen,
SO₂R¹²,
NR¹³SO₂R¹⁴,
C(═W)R¹⁶,
NR¹⁵C(═W)R¹⁷, or
P(═O)OR₄₀R₄₁; wherein
R¹⁰, R¹¹, R¹², R¹³, R¹⁴, R¹⁵, R¹⁶ and R¹⁷, R₄₀ and R₄₁, which may be the same or different, each represent hydrogen, alkyl optionally substituted by optionally substituted aryl, or optionally substituted aryl;
in addition,
R¹⁰ and R¹¹ together with the nitrogen to which they are attached may form a ring;
R¹² may have the same meaning as NR¹⁰R¹¹;
when R₁₇ represents NR¹⁰R¹¹, one of R¹⁰ and R¹¹ may represent hydrogen, COalkyl or optionally substituted COaryl;
R¹⁶ may represent hydroxy, alkoxy, or NR¹⁰R¹¹;
R¹⁷ may represent alkyl optionally substituted by one or more of halogen, alkoxy, or optionally substituted aryl or NR¹⁰R¹¹; and
W is O.

5. The combination of claim 1 wherein:
(i) in the compound of formula I or formula II
A⁵ represents N;
L represents a linker group which is:
O, S or NR¹⁸,
alkylene, alkenylene, or alkynylene, each of which may be optionally interrupted by one or more of O, S, or NR¹⁸, or one or more C—C single, double or triple bonds,
and R¹⁸ represents hydrogen, alkyl, or COR¹⁶.
or (ii) in the compound of formula II
A⁵ represents CH;
L represents a linker group which is:
O, S, or NR¹⁸,
alkylene, alkenylene, or alkynylene, each of which may be optionally interrupted by one or more of O, S, or NR¹⁸, or one or more C—C single, double or triple bonds,
a —N—N— single or double bond,
and R¹⁸ represents hydrogen, alkyl, or COR¹⁶.

6. The combination according to claim 1 in which
when any of the substituents represents alkyl, the alkyl is saturated and has from 1 to 10 carbon atoms; and
when any of the substituents represents aryl, the aryl is an aromatic carbocycle or a 5- to 10-membered aromatic heterocycle containing from 1 to 4 hetero atoms selected from O, S and N.

7. (canceled)

8. The combination according to claim 6, in which the aryl is phenyl, naphthalene, furan, thiophene, pyrrole or pyridine.

9. (canceled)

10. The combination according to claim 1, in which ring B or ring C is a saturated or unsaturated 3 to 10 membered carbocyclic or heterocyclic ring.

11. The combination according to claim 1, in which ring B is a benzene ring, and ring C is a 3-10 membered saturated or unsaturated carbocyclic ring.

12. (canceled)

13. The combination according to claim 1, in which at least one R₁ represents
NR₁₅C(═O)R₁₇;
CONR₁₀R₁₁;
NHCOR₁₇ wherein R₁₇ is:
C₁-C₆ alkyl optionally substituted with one or more halo, phenyl or C₁-C₆ alkoxy,
phenyl optionally substituted with one or more of halo, C₁-C₆ alkyl, C₁-C₆ alkoxy, amino, (C₁-C₆ alkyl)amino di(C₁-C₆ alkyl)amino or phenyl,
CH:CH phenyl,
naphthyl, pyridinyl, thienyl or furanyl;
NR₁₅CONR₁₀R₁₁, wherein R₁₀ and R₁₁, which may be the same or different, are each independently optionally substituted aryl, alkyl or optionally substituted COaryl;
NHCONHR₁₅, wherein R₁₅ is phenyl, C₁-C₆ alkyl or COphenyl optionally substituted with one or more halo;
C₁-C₆ alkyl optionally substituted with phenyl or a 4- to 7-membered saturated or unsaturated heterocycle containing one to two heteroatoms selected from N, S and O; or
COR₁₆, wherein R₁₆ is C₁-C₆ alkoxy, amino, (C₁-C₆ alkyl)amino or di(C₁-C₆ alkyl)amino;
NO₂;
halogen;
amino or (C₁-C₆ alkyl)amino or di(C₁-C₆ alkyl)amino, wherein the C₁-C₆ alkyl is optionally substituted with phenyl or a 5- or 6-membered saturated or unsaturated heterocycle;
NHSO₂(C₁-C₆ alkyl);
NHSO₂phenyl;
SO₂(C₁-C₆ alkyl);
phenyl optionally substituted with one or more C₁-C₆ alkoxy; or
5- to 10-membered saturated or unsaturated mono- or bi-cyclic heterocycle containing from 1 to 3 heteroatoms selected from N, S and O.

14-16. (canceled)

17. The combination according to claim 1 in which one or both of R₁ and R₂ is other than —COOH.

18-22. (canceled)

23. The combination according to claim 1, in which R₃ is a 5- to 10-membered aromatic mono- or bi-cyclic hydrocarbon ring or heterocyclic ring optionally substituted by one to three substituents, which may be the same or different, wherein the heterocyclic ring contains one to three heteroatoms independently selected from N, O and S.

24-25. (canceled)

26. The combination according to claim 23, in which the aromatic ring is benzene, naphthalene, thiophene, furan, pyridine or pyrrole.

27-28. (canceled)

29. The combination according to claim 2, in which the substituent(s) on R³ is R², and R² is independently:
alkyl C₁-C₆, optionally substituted by thienyl or phenoxy, each optionally substituted by halogen,
alkoxy C₁-C₆,
phenyl,
thioalkyl C₁-C₆,
thienyl, optionally substituted by halogen,
NO₂,
CN;
NR₁₀R₁₁, in which R₁₀ and R₁₁, which may be the same or different, each represent hydrogen, or alkyl C₁-C₆, or together with the nitrogen to which they are attached form a 5- to 7-membered ring which may contain one or more additional heteroatoms selected from N, O and S,
halogen,
SO₂R₁₂, in which R₁₂ represents a 5- to 7-membered ring which may contain one or more additional heteroatoms selected from N, O and S, or
NHCOR₁₇, in which R¹⁷ represents
alkyl C₁-C₆, optionally substituted by:
phenyl or halogen, or
phenyl optionally substituted by alkoxy C₁-C₆, carboxy, or halogen, or
a 5 or 6 membered saturated or unsaturated heterocycle, or phenyl or a 5 or 6 membered saturated or unsaturated heterocycle optionally substituted by halogen, alkoxy C₁ to C₆, carboxy or SO₂NR₁₀R₁₁.

30. The combination according to claim 29 in which NR₁₀R₁₁ represents N-pyrrole, N-piperidine, N′—(C₁-C₆ alkyl)-N-piperazine or N-morpholine.

31. The combination of claim 1 comprising the compound of formula II in which A⁵ represents CH, and L represents:
—NH.NH—,
—CH═CH—,
—C≡C— or
—NCOR₁₆ in which R₁₆ represents phenyl or a 5 or 6 membered saturated or unsaturated heterocycle optionally substituted by halogen, alkoxy C₁ to C₆, or carboxy.

32. The combination according to claim 1 in which two of A₁-A₄ represent nitrogen, or one of A₁-A₄ represents nitrogen, or all of A₁-A₄ represents CR₁.

33-34. (canceled)

35. The combination according to claim 1, wherein the compound of formula (I) or (II) is:
N-(2-(4-chlorophenyl)-6-methyl-2H-benzo[d][1,2,3]triazol-5-yl)nicotinamide;
N-(2-(4-chlorophenyl)-6-methyl-2H-benzo[d][1,2,3]triazol-5-yl)isonicotinamide;
N-(2-(4-chlorophenyl)-6-methyl-2H-benzo[d][1,2,3]triazol-5-yl)benzamide;
N-(2-(4-chlorophenyl)-6-methyl-2H-benzo[d][1,2,3]triazol-5-0)-4-methoxybenzamide;
N-(2-(4-chlorophenyl)-6-methyl-2H-benzo[d][1,2,3]triazol-5-yl)-2-methoxybenzamide;
N-(2-(4-chlorophenyl)-6-methyl-2H-benzo[d][1,2,3]triazol-5-yl)thiophene-2-carboxamide;
N-(2-(4-chlorophenyl)-6-methyl-2H-benzo[d][1,2,3]triazol-5-yl)propionamide;
N-(2-(4-chlorophenyl)-6-methyl-2H-benzo[d][1,2,3]triazol-5-yl)butyramide;
N-(2-(4-chlorophenyl)-6-methyl-2H-benzo[d][1,2,3]triazol-5-yl)pentanamide;
N-(2-(4-chlorophenyl)-6-methyl-2H-benzo[d][1,2,3]triazol-5-yl)isobutyramide;
N-(2-(4-chlorophenyl)-6-methyl-2H-benzo[d][1,2,3]triazol-5-yl)furan-2-carboxamide;
N-(2-(4-(diethylamino)phenyl)-6-methyl-2H-benzo[d][1,2,3]triazol-5-yl)nicotinamide;
N-(2-(4-(diethylamino)phenyl)-6-methyl-2H-benzo[d][1,2,3]triazol-5-yl)isonicotinamide;
N-(2-(4-(diethylamino)phenyl)-6-methyl-2H-benzo[d][1,2,3]triazol-5-yl)propionamide;
N-(2-(4-(diethylamino)phenyl)-6-methyl-2H-benzo[d][1,2,3]triazol-5-yl)butyramide;
N-(2-(4-(diethylamino)phenyl)-6-methyl-2H-benzo[d][1,2,3]triazol-5-yl)pentanamide;
N-(2-(4-(diethylamino)phenyl)-6-methyl-2H-benzo[d][1,2,3]triazol-5-yl)isobutyramide;
N-(2-(4-(diethylamino)phenyl)-6-methyl-2H-benzo[d][1,2,3]triazol-5-yl)furan-2-carboxamide;
2-(4-(diethylamino)phenyl)-2H-benzo[d][1,2,3]triazol-5-amine;
N-(2-(4-(diethylamino)phenyl)-2H-benzo[d][1,2,3]triazol-5-yl)nicotinamide;
N-(2-(4-(diethylamino)phenyl)-2H-benzo[d][1,2,3]triazol-5-yl)isonicotinamide;
N-(2-(4-(diethylamino)phenyl)-2H-benzo[d][1,2,3]triazol-5-yl)acetamide;
N-(2-(4-(diethylamino)phenyl)-2H-benzo[d][1,2,3]triazol-5-yl)propionamide;
N-(2-(4-(diethylamino)phenyl)-2H-benzo[d][1,2,3]triazol-5-yl)butyramide;
N-(2-(4-(diethylamino)phenyl)-2H-benzo[d][1,2,3]triazol-5-yl)pentanamide;
N-(2-(4-(diethylamino)phenyl)-2H-benzo[d][1,2,3]triazol-5-yl)isobutyramide;
N-(2-(4-(diethylamino)phenyl)-2H-benzo[d][1,2,3]triazol-5-yl)furan-2-carboxamide;
N-(2-(4-(diethylamino)phenyl)-2H-benzo[d][1,2,3]triazol-5-yl)thiophene-2-carboxamide;
N-(2-(4-(diethylamino)phenyl)-2H-benzo[d][1,2,3]triazol-5-yl)benzamide;
N-(2-(4-(diethylamino)phenyl)-2H-benzo[d][1]triazol-5-yl)-4-methoxybenzamide;
N-(2-(4-(diethylamino)phenyl)-2H-benzo[d][1,2,3]triazol-5-yl)-2-methoxybenzamide;
4-chloro-N-(2-(4-(diethylamino)phenyl)-2H-benzo[d][1,2,3]triazol-5-yl)benzamide;
N-(2-(4-(diethylamino)phenyl)-2H-benzo[d][1,2,3]triazol-5-yl)-4-(dimethylamino)benzamide:
6-methyl-2-(4-morpholinophenyl)-2H-benzo[d][1,2,3]triazol-5-amine;
N-(2-(4-chlorophenyl)-2H-benzo[d][1,2,3]triazol-5-yl)propionamide;
N-(2-(4-chlorophenyl)-2H-benzo[d][1,2,3]triazol-5-yl)butyramide;
N-(2-(4-chlorophenyl)-2H-benzo[d][1,2,3]triazol-5-yl)isobutyramide;
2-(4-chlorophenyl)-2H-benzo[d][1,2,3]triazol-5-amine;
N-(2-(4-chlorophenyl)-2H-benzo[d][1,2,3]triazol-5-yl)acetamide;
2-(4-(piperidin-1-yl)phenyl)-2H-benzo[d][1,2,3]triazol-5-amine;
2-(4-(dimethylamino)phenyl)-2H-benzo[d][1,2,3]triazol-5-amine;
2-(4-(4-methylpiperazin-1-yl)phenyl)-2H-benzo[d][1,2,3]triazol-5-amine;
2-(4-chlorophenyl)-6-methyl-2H-benzo[d][1,2,3]triazol-5-amine;
2-(4-chlorophenyl)-6-(methylsulfonyl)-2H-indazole;
2-(4-chlorophenyl)-6-nitro-2H-indazole;
N-(2-(4-chlorophenyl)-2H-indazol-6-yl)isobutyramide;
2-(4-chlorophenyl)-6-(methylsulfonyl)-2H-benzo[d][1,2,3]triazole 1-oxide;
2-(4-chlorophenyl)-2H-indazole;
2-(4-chlorophenyl)-5-(methylsulfonyl)-2,4-benzo[d][1,2,3]triazole;
2-(3,4-dichlorophenyl)-5-(methylsulfonyl)-2H-benzo[d][1,2,3]triazole;
2-(3′,4′-dichlorophenyl)-5-(ethylsulfonyl)-benzotriazole;
2-(4′-chlorophenyl)-5-(ethylsulfonyl)-benzotriazole;
N-(2-(3,4-Dichlorophenyl)-2H-benzo[d][1,2,3]triazol-5-yl)isobutyramide;
6-(Methylsulfonyl)-2-(naphthalen-2-yl)-2H-benzo[d][1,2,3]triazole 1-oxide;
5-(Methylsulfonyl)-2-(naphthalen-2-yl)-2H-benzo[d][1,2,3]triazole; or
2-(4′-Chlorophenyl)-6-(isopropylsulfonyl)-2H-indazole.

36. The combination of claim 1 wherein the ancillary agent and the compound of formula (I) or (II) are physically associated; or the ancillary agent and the compound of formula (I) or (II) are non-physically associated; or the ancillary agent and the compound of formula (I) or (II) are:
(a) in admixture;
(b) chemically/physicochemically linked;
(c) chemically/physicochemically co-packaged; or
(d) unmixed but co-packaged or co-presented.

37-38. (canceled)

39. The combination of claim 36 wherein the combination comprises: (a) at least one of the two or more compounds together with instructions for the extemporaneous association of the at least one compound to form a physical association of the two or more compounds; or (b) at least one of the two or more compounds together with instructions for combination therapy with the two or more compounds; (c) at least one of the two or more compounds together with instructions for administration to a patient population in which the other(s) of the two or more compounds have been or are being administered; (d) at least one of the two or more compounds in an amount or in a form which is specifically adapted for use in combination with the other(s) of the two or more compounds.

40. A pharmaceutical pack, kit or patient pack comprising the combination of claim 1.

41-43. (canceled)

44. A method for the treatment or prophylaxis of Duchenne muscular dystrophy, Becker muscular dystrophy or cachexia, comprising administering the combination of claim 1.

45. (canceled)

46. The combination of claim 1 wherein the ancillary agent is selected from: (a) an anti-inflammatory agent; (b) a protease inhibitor; (c) a myostatin antagonist; (d) a cytokine or mobilizing agent; (e) a corticosteroid; (f) an anabolic steroid; (g) a TGF-β antagonist; (h) an antioxidant or mitochondrial supporting agent; (i) a dystrophin expression enhancing agent; (j) a gene replacement/repair agent; (k) a cell-based composition; (l) creatine; (m) an anti-osteoporotic agent; (n) an auxiliary utrophin upregulating agent; (o) a cGMP signaling modulator; and (p) a combination of two or more of the foregoing classes (a) to (o).

47. The combination of claim 1 wherein the ancillary agent is a corticosteroid, prednisone, prednisolone or deflazacort.

48-49. (canceled)
